(19) United States
(12) Patent Application Publication
(10) Pub. No.: US 2024/0252107 A1
(43) Pub. Date: Aug. 1, 2024

(54) COMPUTING DEVICE FOR PREDICTING SLEEP STATE ON BASIS OF DATA MEASURED IN SLEEP ENVIRONMENT OF USER

(71) Applicant: Asleep, Seongnam-si, Gyeonggi-do (KR)

(72) Inventors: Dongheon LEE, Seongnam-si, Gyeonggi-do (KR); Junki HONG, Daejeon (KR); Jinhwan JUNG, Daejeon (KR)

(21) Appl. No.: 18/019,155

(22) PCT Filed: Aug. 4, 2021
(86) PCT No.: PCT/KR2021/010273

§ 371 Date: May 31, 2023

(30) Foreign Application Priority Data
Aug. 4, 2020 (KR) 10-2020-0097153
Dec. 10, 2020 (KR) 10-2020-0172298
Dec. 10, 2020 (KR) 10-2020-0172299

(51) Int. Cl. A61B 5/00; A61B 5/01; A61B 5/0205; A61B 5/024; A61B 5/08; A61B 5/11; G16H 10/60; G16H 20/30; G16H 20/60; G16H 50/70

(52) U.S. Cl. CPC A61B 5/4818; A61B 5/0022; A61B 5/01; A61B 5/024; A61B 5/0803; A61B 5/11; A61B 5/4812; A61B 5/4815; A61B 5/7203; A61B 5/7246; A61B 5/7267; A61B 5/7275; G16H 10/60; G16H 20/30; G16H 20/60; G16H 50/70; A61B 5/02055; A61B 2505/07; A61B 2560/0242; A61B 2560/045; A61B 2562/0204; A61B 2562/0271; A61B 2562/029

(57) ABSTRACT

In order to solve the described problem, disclosed is a computing device for predicting a sleep state based on data measured in the sleep environment of a user. The computing device comprises: a processor which receives sleep sensing data of the user and which inputs the sleep sensing data into a sleep assessment model to predict sleep analysis information about the user; a memory for storing program codes that can be executed by the processor; and a network unit for transmitting/receiving data to/from a user terminal, wherein the sleep sensing data includes information on breathing about the user, which is acquired for a predetermined time period in relation to the sleep environment of the user, and the sleep analysis information can include apnea severity information about the degree at which apnea of the user occurs and/or disease prediction information about the possibility of disease occurrence.

### TECHNICAL FIELD

The present disclosure relates to providing sleep state information based on a user's sleep data acquired from a sleep environment, and more particularly, is intended to provide analysis information and healthcare information corresponding to a sleep state of a user using an artificial neural network.

### BACKGROUND ART

There are various ways to maintain and improve health, such as exercise, diet, etc., but it is most important to manage sleep that accounts for more than 30% of the time of the day. However, despite replacement of simple labor by machines and leisure of life, modern people cannot get a sound sleep due to irregular eating habits, irregular lifestyles, and stress, and suffer from sleep disorders such as insomnia, hypersomnia, sleep apnea, nightmares, night terrors, sleepwalking, etc.

According to the Korea's National Health Insurance Service, the number of sleep disorder patients in Korea increased by an average of about 8% per year from 2014 to 2018, and in 2018, about 570,000 patients were treated for sleep disorders in Korea.

In particular, one out of six Korean adults suffers from sleep apnea, the most common but dangerous sleep disorder. Such sleep apnea not only interferes with sound sleep but is also considered a major cause of heart disorders, mental disorders, brain disorders, etc.

As sound sleep is considered an important factor that affects physical or mental health, interest in sound sleep is on the rise. However, to treat sleep disorders, it is necessary to visit a specialized medical institution in person and additionally pay for an examination. Additionally, it is difficult to continuously manage sleep disorders. Accordingly, users' efforts for treatment are insufficient.

For this reason, Korean Patent Application Publication No. 10-2003-0032529 discloses a sleep induction apparatus and method for receiving physical information on a user and outputting vibrations and/or ultrasonic waves in a frequency band detected through repeated learning according to a physical state of the user in a sleep to optimally induce sound sleep.

However, the related art has a risk of degrading the quality of sleep due to inconvenience caused by wearable equipment, and it is necessary to periodically manage (e.g., charging and the like) the equipment. Additionally, the related art only provides information for inducing optimal sleep, and the correlation between information obtainable from sleep and associated disorders is not taken into consideration. Accordingly, it is not possible to provide prediction information on a probability of disorder occurrence in the future, and neither medical diagnosis nor information transmission is possible.

Consequently, in the corresponding field, there may be demands for a computing device that can continuously diagnose a sleep disorder by collecting data related to a user's sleep environment and can also predict probabilities of developing disorders associated with a sleep disorder (e.g., heart disorders, mental disorders, and brain disorders).

### DISCLOSURE OF THE INVENTION

### Technical Problems

The present disclosure is directed to providing information on a sleep state based on data measured in a sleep environment of each individual user, predicting a probability of developing a sleep disorder and accompanying disorders associated with the sleep disorder, and providing medical diagnosis information to the user.

Objects of the present disclosure are not limited to those described above, and other objects which are not described will be clearly understood by those of ordinary skill in the art from the following descriptions.

### Technical Solutions

One embodiment of the present disclosure provides a computing device for predicting a sleep state based on data measured in a sleep environment of a user. The computing device includes a processor configured to receive sleep sensing data of the user and predict sleep analysis information on the user by inputting the sleep sensing data to a sleep assessment model, a memory configured to store program codes executable by the processor, and a network unit configured to transmit or receive data to or from a user terminal. The sleep sensing data includes information on breathing of the user obtained during a predetermined time period with regard to the sleep environment of the user. The sleep analysis information includes at least one of apnea severity information on a degree of apnea occurrence of the user and disorder prediction information on a probability of disorder occurrence of the user.

According to an embodiment, the computing device may further include a sensor unit configured to acquire the sleep sensing data of the user, and the sensor unit may include at least one transmission module configured to transmit a radio wave of a specific frequency and a reception module configured to receive a reflected wave generated in response to the radio wave of the specific frequency, and may acquire the sleep sensing data from the user in a contactless manner by detecting a phase difference or a frequency change according to a travel distance of the reflected wave.

According to an embodiment, the processor may receive a sleep diagnosis dataset including a plurality of pieces of sleep diagnosis data each corresponding to a plurality of users, generate training input datasets by extracting information on the users' respiration, heart rates, and movement during the predetermined time period from each of the plurality of pieces of sleep diagnosis data, generate training output datasets by extracting at least one of information on apnea indices, information on sleep states, and information on whether a disorder has occurred from each of the plurality of pieces of sleep diagnosis data, generate labeled training datasets by matching the training input datasets with the training output datasets, and generate the sleep assessment model by training one or more network functions using the labeled training datasets.

According to an embodiment, to generate the sleep assessment model, the processor may input each of the training input datasets to the one or more network functions, derive errors by comparing output data predicted through the one or more network functions with the training output datasets corresponding to labels of the training input datasets, adjust weights of the one or more network functions based on the errors using a backpropagation method, determine whether to stop training using verification data when the one or more network functions are trained for predetermined epochs or more, and test performance of the one or more network functions using test datasets to determine whether to activate the one or more network functions.

According to an embodiment, the sleep assessment model may be a model for predicting the user's sleep state and probability of disorder occurrence based on the sleep sensing data and may include one or more network functions, which may include a dilated Convolutional Neural Network (CNN) for strengthening a long-term relationship without loss of input data.

According to an embodiment, the dilated CNN may strengthen the long-term relationship by expanding a receptive field of a filter related to inputs of one or more network functions and maintain lengths of the inputs and outputs of the one or more network functions by adding zero-padding to the filter related to the inputs.

According to an embodiment, the disorder prediction information may include prediction information on at least one of sleep disorders, mental disorders, brain disorders, and cardiovascular disorders, and the processor may derive a correlation between the sleep sensing data and the disorder prediction information based on a variation of the disorder prediction information that is output by changing the information on breathing for the predetermined time period included in the sleep sensing data of the user input to the sleep assessment model.

According to an embodiment, the sleep sensing data may include information on movement and information on heart rate for the predetermined time period, and the sleep analysis information may include sleep stage information on changes in one or more sleep states over time with respect to the sleep environment of the user.

According to an embodiment, the computing device may further include a sensor unit including one or more environment sensing modules for acquiring indoor environment information including information on at least one of a body temperature of the user, an indoor temperature, an indoor humidity, indoor sound, and indoor brightness, and the processor may generate sleep degradation factor information based on the sleep stage information and the indoor environment information.

According to an embodiment, the processor may identify a first time point at which quality of the user's sleep is degraded based on the sleep stage information, identify a singularity related to a variation of the indoor environment information corresponding to the first time point, and generate the sleep degradation factor information based on the identified singularity.

According to an embodiment, the processor may identify the singularity related to volatility of the indoor environment information based on whether a variation of each of the at least one piece of information included in the indoor environment information exceeds a predetermined threshold variation corresponding to the first time point.

According to an embodiment, the computing device may further include an indoor environment setup unit configured to adjust at least one of the temperature, the humidity, the sound, and the brightness to setup an indoor environment related to the sleep environment of the user, and the processor may generate an environment control signal for controlling the indoor environment setup unit based on the sleep degradation factor information.

According to an embodiment, the processor may generate healthcare information for improving the sleep environment and health of the user based on sleep assessment information and determine to transmit the healthcare information to the user terminal of the user, and the health care information may include at least one of eating habit information, exercise amount information, and optimal sleep environment information.

Another embodiment of the present disclosure provides a method of predicting a sleep state and disorder based on data measured in a sleep environment of a user, which is performed by at least one processor of a computing device. The method includes receiving, by the processor, sleep sensing data of the user, inputting, by the processor, the sleep sensing data to a sleep assessment model to predict sleep analysis information on the user, and determining, by the processor, to transmit the sleep analysis information to a user terminal of the user.

Another aspect of the present disclosure provides a computer program stored on a computer-readable recording medium. When the computer program is executed by at least one processor, the computer program performs, to predict a sleep state based on data acquired in a sleep environment of a user, the following operations of receiving sleep sensing data of the user, inputting, by the processor, the sleep sensing data to a sleep assessment model to predict sleep analysis information on the user, and determining, by the processor, to transmit the sleep analysis information to a user terminal of the user.

Other details of the present disclosure are included in the detailed description and accompanying drawings.

### Effects of the Invention

The present disclosure has been devised in response to the background art described above and can provide information on a sleep disorder based on data measured in a sleep environment of each individual user and also provide medical diagnosis information to the user by predicting a probability of developing a sleep disorder and an associated disorder, thereby improving efficiency in healthcare.

Effects of the present disclosure are not limited to that described above, and other effects which are not described will be clearly understood by those of ordinary skill in the art from the following descriptions.

### BRIEF DESCRIPTION OF THE DRAWINGS

FIG. 1 is a conceptual diagram illustrating a system through which various aspects of a computing device for providing sleep analysis information based on sleep environment sensing data related to a sleep environment of a user according to one embodiment of the present disclosure are implemented.

FIG. 2 is a block diagram of the computing device for providing sleep analysis information based on sleep environment sensing data related to a sleep environment of a user according to one embodiment of the present disclosure.

FIG. 3 is an example view of a computing device that acquires sleep sensing data related to a sleep environment of a user according to one embodiment of the present disclosure.

FIG. 4 is an example diagram illustrating a process of training a sleep assessment model according to one embodiment of the present disclosure.

FIG. 5 is a graph illustrating sleep sensing data according to one embodiment of the present disclosure.

FIG. 6 is a diagram illustrating a process of inputting sleep sensing data to a sleep analysis model according to one embodiment of the present disclosure.

FIG. 7 is a diagram illustrating an output process of the sleep analysis model according to one embodiment of the present disclosure.

FIG. 8 is an example diagram illustrating a transmission module and a reception module provided to acquire object state information according to one embodiment of the present disclosure.

FIG. 9 is an example diagram illustrating a transmission module and a reception module provided to acquire object state information according to another embodiment of the present disclosure.

FIG. 10 is a flowchart illustrating a preprocessing process for channel state information according to one embodiment of the present disclosure.

FIG. 11 is a flowchart illustrating a method of providing sleep analysis information based on sleep sensing data related to a sleep environment of a user according to one embodiment of the present disclosure.

FIG. 12 is a flowchart illustrating a method of providing sleep-related analysis information according to one embodiment of the present disclosure.

FIG. 13 is a flowchart illustrating a method of acquiring object state information based on wireless communication according to one embodiment of the present disclosure.

FIG. 14 is a schematic diagram illustrating one or more network functions according to one embodiment of the present disclosure.

### BEST MODE FOR CARRYING OUT THE INVENTION

Advantages and features of the present disclosure and a method of achieving them will become apparent from embodiments which are described in detail below with reference to the accompanying drawings. However, the present disclosure is not limited to the embodiments disclosed below and may be implemented in a variety of different forms. The embodiments are provided to make the disclosure of the present disclosure complete and fully inform those skilled in the technical field to which the present disclosure pertains of the scope of the present disclosure. The present disclosure is only defined by the scope of the claims.

Terminology used herein is for the purpose of describing embodiments only and is not intended to limit the present disclosure. As used herein, the singular forms include the plural forms as well unless the context clearly indicates otherwise. The terms “comprise” and/or “comprising” used herein do not preclude the presence or addition of one or more components other than stated components. Throughout the specification, like numbers refer to like components, and “and/or” includes any one or all possible combinations of stated components. Although “first,” “second,” etc. are used to describe various components, the components are not limited by the terms. These terms are only used to distinguish one component from other components. Accordingly, it is apparent that a first component described below may be a second component without departing from the technical spirit of the present disclosure.

Unless otherwise defined, all terms (including technical and scientific terms) may be used as meanings commonly understood by those skilled in the technical field to which the present disclosure pertains. Also, unless clearly defined otherwise, all terms defined in generally used dictionaries are not to be ideally or excessively interpreted.

The term “unit” or “module” used herein means a software or hardware component, such as a field-programmable gate array (FPGA) and an application-specific integrated circuit (ASIC), and a “unit” or “module” performs certain roles. However, a “unit” or “module” is not limited to software or hardware. A “unit” or “module” may be configured to be in an addressable storage medium or may be configured to run on one or more processors. Accordingly, as an example, a “unit” or “module” may include components, such as software components, object-oriented software components, class components, and task components, as well as processors, functions, attributes, procedures, subroutines, segments of program code, drivers, firmware, micro-code, circuits, data, databases (DBs), data structures, tables, arrays, and variables. Functions provided in components and “units” or “modules” may be combined into a smaller number of components and “units” or “modules” or subdivided into additional components and “units” or “modules.”

In the specification, a computer refers to any type of hardware device including at least one processor and may be understood as encompassing software components operating in a corresponding hardware device according to embodiments. For example, a computer may be understood as encompassing, but is not limited to, all of a smartphone, a tablet personal computer (PC), a desktop computer, a notebook computer, and a user client and applications running on each of the devices.

Those of ordinary skill in the art should additionally appreciate that various illustrative logical blocks, configurations, modules, circuits, means, logics, and algorithm steps described with regard to embodiments disclosed herein may be implemented as electronic hardware, computer software executed by a processor, or combinations thereof. Various illustrative components, blocks, configurations, means, logics, modules, circuits, and steps have been described above generally in terms of their functionality. Whether such functionality is implemented using hardware or software depends on a particular application and design constraints imposed on the overall system. Skilled engineers may implement the described functionality in a variety of ways for each particular application. However, such implementation decisions should not be interpreted as causing a departure from the scope of the present disclosure.

Hereinafter, embodiments of the present disclosure will be described in detail with reference to the accompanying drawings.

Each step described herein is described as being performed by a computer, but a subject of each step is not limited thereto, and at least some of the steps may be performed in different devices according to embodiments.

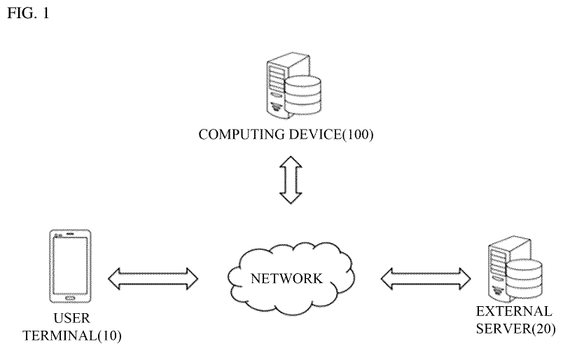

FIG. 1 is an example diagram illustrating a system through which various aspects of a computing device for predicting a sleep state based on data measured in a sleep environment of a user according to one embodiment of the present disclosure are implemented.

The system according to embodiments of the present disclosure may include a computing device 100, a user terminal 10, an external server 20, and a network. The computing device 100, the user terminal 10, and the external server 20 according to embodiments of the present disclosure may transmit or receive data for the system according to embodiments of the present disclosure to or from each other through the network.

The network according to embodiments of the present disclosure may employ various wired communication systems such as a public switched telephone network (PSTN), an x digital subscriber line (xDSL), a rate adaptive DSL (RADSL), a multi-rate DSL (MDSL), a very high speed DSL (VDSL), a universal asymmetric DSL (UADSL), a high bit rate DSL (HDSL), a local area network (LAN), etc.

Also, the network proposed herein may employ various wireless communication systems such as a code division multi-access (CDMA) system, a time division multi-access (TDMA) system, a frequency division multi-access (FDMA) system, an orthogonal frequency division multi-access (OFDMA) system, a single carrier-FDMA (SC-FDMA) system, and other systems.

The network according to embodiments of the present disclosure may be configured using any communication method, such as wired communication, wireless communication, etc., and may be various communication networks such as a personal area network (PAN), a wide area network (WAN), etc. Also, the network may be the known World Wide Web (WWW) and may employ a wireless transmission technology used for short-range communication such as infrared data association (IrDA) or Bluetooth. Technologies described herein may be used not only in the foregoing networks but also in other networks.

According to an embodiment of the present disclosure, the user terminal 10 is a terminal that may receive information on a user's sleep by exchanging information with the computing device 100, and may indicate a terminal carried by the user. For example, the user terminal 10 may be a terminal related to a user who wants to improve his or her health through information on his or her sleeping habits. Through the user terminal 10, the user may receive information on his or her sleep, prediction information on a probability of associated disorder occurrence, information for reducing a probability of disorder occurrence, information for improving his or her health, etc.

According to an embodiment of the present disclosure, the user terminal 10 is a terminal that may receive information on the user's sleep through information exchange with the computing device 100 and may indicate a terminal carried by the user. Also, the user terminal 10 may be a terminal that may receive object state information on an object present in an area through information exchange with the computing device 100.

For example, the user terminal 10 may be a terminal related to a user who wants to improve his or her health through information on his or her sleeping habits. Also, the user terminal 10 may be a terminal related to an examiner (e.g., a medical specialist) who provides a diagnosis result to the user (e.g., an examinee). When the user terminal 10 is a terminal related to the examiner who provides a checkup result (e.g., a polysomnography result) to the examinee, the user terminal 10 may be used as a medical auxiliary terminal for interpreting the checkup result of the examinee through analysis information acquired from the computing device 100.

The user may receive sleep stage information corresponding to each time point in a sleep environment, prediction certainty information corresponding to sleep stage information on each time point, etc. through the user terminal 10. Also, the user may receive bioinformation on his or her sleep (e.g., information on breathing, information on heart rate, motion information, etc.), information about whether he or she has a disorder obtained through analysis based on the bioinformation, etc. through the user terminal 10. The user terminal 10 includes a display and thus can receive an input of the user and provide any form of output to the user.

The user terminal 10 may be referred to as, but is not limited to, any device capable of using a wireless mechanism such as a user equipment (UE), a mobile, a PC capable of wireless communication, a cell phone, a kiosk, a cellular phone, a cellular, a cellular terminal, a subscriber unit, a subscriber station, a mobile station, a terminal, a remote station, a personal digital assistant (PDA), a remote terminal, an access terminal, a user agent, a cellular phone, a wireless phone, a session initiation protocol (SIP) phone, a wireless local loop (WLL) station, a portable device having a wireless access function, and a wireless model. Also, the user terminal 10 may be referred to as, but is not limited to, an arbitrary device capable of using a wire access mechanism such as a wired fax, a PC equipped with a wired model, a wired phone, a terminal capable of wired communication, etc.

According to an embodiment of the present disclosure, the external server 20 may be a server that stores health checkup information, sleep-checkup information, etc. of a plurality of users. For example, the external server 20 may be at least one of a hospital server and an information server and may store information on a plurality of polysomnography records, electronic health records, electronic medical records, etc. For example, a polysomnography record may include information on brain waves, eye movements, muscle movements, respiration, electrocardiogram, etc. of a sleep-checkup subject during a sleep and information on a sleep-checkup result (e.g., an apnea, a somnipathy, etc.) corresponding to the information. Also, a polysomnography record may include information about whether a checkup subject suffers from a disorder, for example, information that the checkup subject has at least one of a heart disorder (ischemic myocardial infarction, heart attack, arrhythmia, etc.), a brain disorder (hemorrhagic stroke, ischemic stroke, transient ischemia, cerebral infarction, etc.), and a mental disorder (depression, anxiety disorder, bipolar disorder, panic disorder, dementia, delusional disorder, etc.). Information stored in the external server 20 may be used as training data for training a neural network, verification data, and test data in the present disclosure.

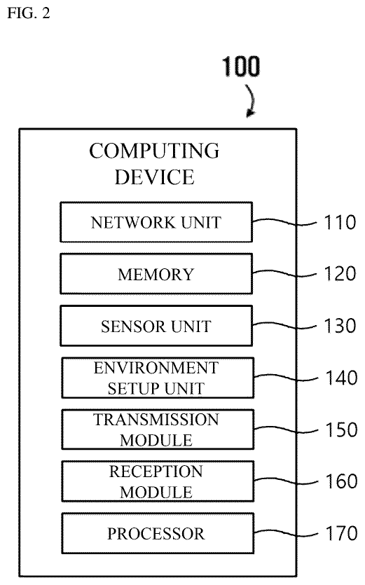

The computing device 100 of the present disclosure may receive health checkup information, sleep-checkup information, etc. from the external server 20 and generate training datasets based on the information. The computing device 100 may train a sleep assessment model including one or more network functions using the training datasets to generate a sleep assessment model for predicting sleep analysis information corresponding to a user. Also, the computing device 100 may train a sleep analysis model including one or more network functions using the training datasets to generate a sleep analysis model for predicting sleep analysis information corresponding to sleep sensing data of a user. A configuration for generating training datasets for neural network training and a training method employing training datasets according to the present disclosure will be described in detail below with reference to FIG. 2.

According to an embodiment of the present disclosure, the external server 20 may be a server system that provides an intensive processing function in a short-range communication network. The external server 20 may monitor or control an overall network, such as control over arbitrary functions, data management, etc. related to the present disclosure, or help in connecting to another network through a mainframe or public network or sharing software resources, such as data, programs, or files, or hardware resources such as a model, a fax, a printer, other devices, etc. The external server 20 may indicate a computer for storing information that is contained in a hard disk thereof in a special format to be exposed to the outside. In general, various information may be managed by the external server 20, and general users may access the external server 20 using their external devices to use information provided by the external server 20. In the present disclosure, the external server 20 may control, store, or transmit or receive information to share the information with the user terminal 10 and the computing device 100.

The external server 20 of the present disclosure may exchange information with another external server by communicating with the other external server. Also, the external server 20 may store arbitrary information/data in a DB, a computer-readable recording medium, etc. The computer-readable recording medium may include a computer-readable storage medium and a computer-readable communication medium. The computer readable storage medium may include any type of storage medium in which a program and data is stored to be read by a computer system. According to an aspect of the present invention, the computer-readable storage medium may include a read only memory (ROM), a random access memory (RAM), a compact disc (CD)-ROM, a digital video disk (DVD)-ROM, magnetic tape, a floppy disk, an optical data storage device, etc. Also, the computer-readable communication medium may include a medium implemented in the form of carrier waves (e.g., transmission through the Internet). Additionally, such media may be distributed throughout a system connected through a network to store computer-readable codes and/or commands in a distributed manner.

The external server 20 may be a digital device which includes a processor and a memory and has computing power, such as a laptop computer, a notebook computer, a desktop computer, a web pad, or a mobile phone. The external server 20 may be a web server that processes service. The foregoing types of servers are merely examples, and the present disclosure is not limited thereto.

According to an embodiment of the present disclosure, the computing device 100 may acquire sleep sensing data related to a sleep state of a user and generate sleep analysis information on the user's sleep state based on the sleep sensing data. Specifically, the computing device 100 may generate a sleep assessment model for outputting sleep analysis information based on sleep sensing data by training one or more network functions using labeled training datasets. In other words, the computing device 100 may acquire sleep sensing data related to a user's sleep environment and process the sleep sensing data as an input to the sleep assessment model, which is a trained neural network model, to generate sleep analysis information.

In this case, the sleep analysis information provided by the computing device 100 may include at least one of apnea severity information on a degree of apnea occurring during the user's sleep, sleep stage information on changes in the sleep state, and disorder prediction information about a probability of disorder occurrence. In other words, the computing device 100 may provide information on sleep disorders and information for identifying a sleep stage based on data measured in a sleep environment of each individual user. At the same time, the computing device 100 may predict a probability of occurrence of a disorder associated with a sleep disorder and provide medical diagnosis information to the user, thereby increasing efficiency in healthcare.

Also, the sleep analysis information provided by the computing device 100 may include prediction information on sleep stages during the user's sleep. For example, the sleep analysis information may include sleep stage information that the user's sleep corresponds to at least one of one or more sleep stages. The one or more sleep stages may include, for example, wake (a non-sleep state), Non rapid eye movement (REM) 1 (N1), Non REM 2 (N2), Non REM 3 (N3), and REM sleep. As a specific example, the sleep analysis information may include information that the user's sleep stage at a first time point corresponds to REM sleep.

Additionally, the sleep analysis information may include prediction certainty information on each time point related to the user's sleep environment. The prediction certainty information may be information on accuracy in predicting sleep stage information which is output (or predicted) through the sleep analysis model according to a specific time point. For example, the sleep analysis information may include prediction information that the user's sleep stage at the first time point corresponds to REM sleep and information that prediction certainty information on reliability of the prediction information is “80.” For example, a larger value of prediction certainty information may represent that accuracy (or reliability) of a sleep stage predicted through the artificial neural network is higher. In other words, the computing device 100 may provide prediction information on sleep stages for each time points during the user's sleep and further provide prediction certainty information with respect to each of the prediction information on sleep stages. In this case, the prediction certainty information may be used as an indicator for determining how reliable an output (i.e., prediction information on a sleep stage) of the artificial neural network (i.e., the sleep analysis model). In other words, prediction certainty information on prediction of sleep stage for each time point is provided together with sleep stage information such that sleep stage information can be reliably used.

According to an embodiment of the present disclosure, the computing device 100 may generate feature map information based on the sleep analysis information. The feature map information may be information obtained by visualizing data that has influence on a process of predicting the sleep analysis information output by the sleep analysis model. Specifically, the computing device 100 may acquire an attention weight for sleep analysis information according to sleep sensing data of a specific time point through one or more attention modules. In this case, the one or more attention modules may be modules that allow learning of a matching relationship between inputs and outputs of the sleep analysis model. In a neural network training process, the one or more attention modules may emphasize an element to be focused on between input data and output data when an attention weight is given to each element of the time-series input data (i.e., sleep sensing data). In other words, the one or more attention modules may be modules that generate information about which input element has the highest correlation with an output value of the sleep analysis model.

As a specific example, from sleep sensing data of the user acquired at a first time point (e.g., initial one minute of the user's sleep after going to bed), the computing device 100 may generate prediction information that a sleep stage of the user during the one minute corresponds to N3 sleep stage. Also, the computing device 100 may acquire an attention weight of each input element for sleep analysis information (i.e., the prediction information that the sleep stage of the user at the time point corresponds to N3 sleep stage) related to the first time point from the one or more attention modules. The computing device 100 may generate feature map information by visualizing the acquired element-specific attention weights.

In other words, the computing device 100 may visualize prediction procedure for a process of producing prediction information on time-series input data (i.e., sleep sensing data) by using at least one of the Attention modules. In other words, which signal (i.e., which sleep sensing data) plays an important role in determining a sleep stage of each time point may be visualized and provided through attention weight values. For example, the feature map information may be generated to provide visualized information in a meaningful form by differently displaying pixels based on signals or information that attracts attention through attention weights.

In general, it is difficult to understand an internal algorithm of an artificial neural network. In other words, it is difficult to know on what basis an output related to input data is generated. This acts as a risk resulting from uncertainty of the artificial neural network, and thus it may be difficult to use the artificial neural network in an actual medical environment.

The computing device 100 of the present disclosure visualizes a pattern of a signal that plays an important role in predicting a sleep stage at each time point and provides the visualized pattern, and thus it is possible to visualize on what basis a neural network model makes a prediction or determination. Accordingly, it is possible to precisely verify validity of prediction information output by the sleep analysis model of the present disclosure such that collaborative synergy with a user (e.g., a medical specialist) can be maximized.

According to another embodiment of the present disclosure, the computing device 100 may acquire object state information on an object. In the present disclosure, objects may be various objects that move in an area. For example, the object may be a user present in an area. Acquiring object state information may represent monitoring movement or a bio-signal of the object. For example, acquiring object state information may represent acquiring at least one of information on movement, information on heart rate, and information on breathing of the user present in an area. The above-described details of an object and object state information are merely examples, and the present disclosure is not limited thereto. In other words, according to various implementation forms, objects may further include various things, animals, etc. in addition to people.

Specifically, the computing device 100 may acquire channel state information from a wireless signal and acquire object state information on an object present in an area based on the acquired channel state information. In an embodiment, wireless signals may include an orthogonal frequency division multiplexing (OFDM) signal. Such a wireless signal may be transmitted to an area in which the object is present through a transmission module 150 and received through a reception module 160. For example, the transmission module 150 may be implemented through a WiFi transmitter, and the reception module 160 may be implemented through a notebook computer, a smartphone, a tablet PC, etc. In other words, it may be possible to acquire object state information with high reliability through relatively inexpensive equipment.

More specifically, the computing device 100 may include the transmission module 150 that transmits a wireless signal in one direction in which an object is present and the reception module 160 that is at a predetermined separation distance away from the transmission module 150 and receives the wireless signal transmitted by the transmission module 150. The wireless signal is an OFDM signal and thus may be transmitted or received through a plurality of subcarriers. For example, the wireless signal may be a WiFi-based OFDM signal.

The computing device 100 may acquire channel state information based on the wireless signal transmitted by the transmission module 150 and the wireless signal received by the reception module 160. The channel state information may be information representing a characteristic of a channel through which the wireless signal has passed. In this case, the channel through which the wireless signal has passed may indicate an area between the transmission module 150 and the reception module 160 (i.e., the predetermined separation distance) and mean an area in which an object is active or present. For example, the predetermined separation distance between the transmission module 150 and the reception module 160 may indicate an area in which the user goes to sleep.

In other words, the wireless signal transmitted by the transmission module 150 may pass through the specific channel (i.e., an area in which the user is present) and may be received by the reception module 160. In this case, the wireless signal may be transmitted through a plurality of subcarriers each corresponding to multiple paths. Accordingly, the wireless signal received by the reception module 160 may be a signal to which the movement of the object is reflected. In other words, the computing device 100 may acquire the channel state information on the channel characteristic that the wireless signal has experienced while passing through the channel from the received wireless signal. The channel state information may have an amplitude and a phase.

Also, the computing device 100 may acquire object state information on the object using the acquired channel state information. The object state information may include information on movement and a bio-signal of the object. Specifically, the computing device 100 may identify a rotation period on a complex plane based on the channel state information. For example, when the channel state information is repeatedly acquired, a displayed value of the channel state information may vibrate counterclockwise on the complex plane according to periodic movement of the object (e.g., respiration). As an example, according to the amount of movement, the displayed value of the channel state information may draw a circle or an arc on the complex plane while vibrating. The computing device 100 may identify a rotation period on the complex plane through the repeatedly acquired channel state information and acquire object state information based on the identified rotation period. In other words, the computing device 100 may identify a rotation period related to movement of the object on the complex plane through channel state information acquired in time series and acquire object state information through the rotation period. For example, the computing device 100 may identify a rotation period related to the user's breathing through the channel state information and acquire a bio-signal related to the user's breathing through the rotation period.

In other words, the computing device 100 may acquire object state information by monitoring the object based on wireless communication. In this case, the object state information may relate to movement or a bio-signal of the user. In other words, the computing device 100 may acquire state information on movement and a bio-signal of the user without any contact with the user's body.

As described above, the computing device 100 of the present disclosure may acquire object state information on the object through the transmission module in a WiFi transmission device and a reception device implemented in a notebook computer, a smartphone, a tablet PC, etc. In other words, it is possible to acquire object state information with high accuracy through relatively inexpensive equipment. In other words, even without expensive equipment for measuring movement or a bio-signal of an object (e.g., a blood pressure gauge, a pulsimeter, etc.), the computing device 100 provides various object state information on the object, and thus monitoring can be provided with easy access. As a specific example, the computing device 100 of the present disclosure may be implemented in a WiFi transmission device and a tablet PC to monitor information on breathing of a patient at all times. Further, object state information according to the present disclosure may be used in a test that involves acquiring bio-information, such as information on breathing, information on heart rate, etc., (e.g., polysomnography). A detailed configuration of the computing device 100 of the present disclosure and effects of the configuration will be described in detail below with reference to FIG. 2.

Also, the computing device 100 may be a terminal or server and may include any type of device. The computing device 100 may be a digital device which includes a processor and a memory and has computing power, such as a laptop computer, a notebook computer, a desktop computer, a web pad, or a mobile phone. The computing device 100 may be a web server that processes service. The foregoing types of servers are merely examples, and the present disclosure is not limited thereto.

According to an embodiment of the present disclosure, the computing device 100 may be a server that provides a cloud computing service. More specifically, the computing device 100 is a server that provides a service of cloud computing, as a type of Internet-based computing, of processing information through another computer connected to the Internet rather than a computer of a user. The cloud computing service may be a service for storing data in the Internet and enabling a user to use necessary data or a necessary program anytime and anywhere through Internet access without installing the necessary data or program on his or her computer. According to the cloud computing service, it is possible to easily share and transmit data stored in the Internet with simple manipulations and clicks. Also, the cloud computing service may not only simply store data in a server on the Internet but may also enable a user to perform a desired task using functions of application programs provided in a web even without installing a program and enable several people to simultaneously perform a task while sharing a document. Further, the cloud computing service may be implemented in at least one form among infrastructure as a service (IaaS), platform as a service (PaaS), software as a service (SaaS), a virtual machine (VM)-based cloud server, and a container-based cloud server. In other words, the computing device 100 of the present disclosure may be implemented in at least one form among the foregoing cloud computing services. The foregoing cloud computing services are merely examples, and any platform for building a cloud computing environment of the present disclosure may be included.

A method in which the computing device 100 provides sleep analysis information based on sleep environment sensing data or acquires object state information based on wireless communication will be described in detail below with reference to FIG. 2.

FIG. 2 is a block diagram of the computing device for providing sleep analysis information based on sleep environment sensing data related to a sleep environment of a user according to one embodiment of the present disclosure.

As shown in FIG. 2, the computing device 100 may include a network unit 110, a memory 120, a sensor unit 130, an environment setup unit 140, the transmission module 150, the reception module 160, and a processor 170. The foregoing components included in the computing device 100 are exemplary, and the scope of the present disclosure is not limited thereto. In other words, according to implementation forms of embodiments of the present disclosure, additional components may be included, or some of the foregoing components may be omitted.

According to an embodiment of the present disclosure, the computing device 100 may include the network unit 110 that transmits or receives data to or from the user terminal 10 and the external server 20. The network unit 110 may transmit or receive data for performing a method of providing sleep analysis information based on sleep environment sensing data according to an embodiment of the present disclosure, data for performing a method of acquiring object state information based on wireless communication, etc. with another computing device, a server, etc. In other words, the network unit 110 may provide a communication function between the computing device 100 and the user terminal 10 and the external server 20. For example, the network unit 110 may receive sleep-checkup records and electronic health records of a plurality of users from a hospital server. Additionally, the network unit 110 may allow information transfer between the computing device 100 and the user terminal 10 and the external server 20 by calling a procedure to the computing device 100.

The network unit 110 according to an embodiment of the present disclosure may employ one of various wired communication systems such as a PSTN, an xDSL, a RADSL, an MDSL, a VDSL, a UADSL, an HDSL, a LAN, etc.

The network unit 110 proposed in the present disclosure may employ one of various wireless communication system such as a CDMA system, a TDMA system, an FDMA system, an OFDMA system, an SC-FDMA system, and other systems.

In the present disclosure, the network unit 110 may be configured in any communication form, such as wired communication, wireless communication, etc., and configured for various communication networks such as a PAN, a WAN, etc. Also, the network may be the known WWW and may employ a wireless transmission technology used for short-range communication such as IrDA or Bluetooth. Technologies described herein may be used not only in the foregoing networks but also in other networks.

According to an embodiment of the present disclosure, the memory 120 may store a computer program for performing a method of providing sleep analysis information according to an embodiment of the present disclosure, and the stored computer program may be read and executed by the processor 170. Also, the memory 120 may store any form of information generated or determined by the processor 170 and any form of information received by the network unit 110. Also, the memory 120 may store data related to the user's sleep. For example, the memory 120 may temporarily or permanently store input or output data (e.g., sleep sensing data related to a sleep environment of the user, sleep analysis information, health improvement information, a wireless signal, channel state information, object state information, etc.).

According to the embodiment of the present disclosure, the memory 120 may include at least one type of storage medium among a flash memory type storage medium, a hard disk type storage medium, a multimedia card micro type storage medium, a card type memory (e.g., a secure digital (SD) or extreme digital (XD) memory or the like), a RAM, a static random access memory (SRAM), a ROM, an electrically erasable programmable read-only memory (EEPROM), a programmable read-only memory (PROM), a magnetic memory, a magnetic disk, and an optical disk. The computing device 100 may operate in connection with a web storage that performs the storage function of the memory 120 on the Internet. The above descriptions of the memory are merely examples, and the present disclosure is not limited thereto.

According to an embodiment of the present disclosure, the computing device 100 may acquire sleep sensing data using the sensor unit 130. The sensor unit 130 may acquire sleep sensing data related to a sleep environment of the user. The sleep sensing data may include the user's information on breathing acquired during a predetermined time period with regard to the sleep environment of the user. Also, the sleep sensing data may further include information on movement and information on heart rate of the user acquired during the predetermined time period according to the sleep environment of the user. In other words, the sensor unit 130 may include a sensor module for acquiring at least one of information on breathing, information on movement, and information on heart rate from the user with regard to the sleep environment of the user.

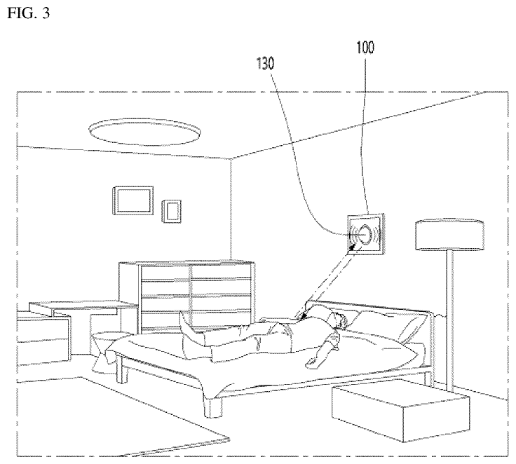

According to an embodiment of the present disclosure, the sensor unit 130 may include one or more transmission module that transmits a radio wave of a specific frequency and a reception module that receives a reflected wave generated in response to the radio wave of the specific frequency (e.g., a microwave). In this case, the sensor unit 130 may acquire sleep sensing data from the user in a contactless manner by detecting a phase difference or a frequency change according to the travel distance of the reflected wave corresponding to the radio wave transmitted by the transmission module. For example, when the radio wave transmitted by the transmission module hits an object and is reflected, the phase difference or frequency may vary. For example, when the object comes closer to the transmission module, the frequency of the reflected wave may increase, and when the object moves away from the transmission module, the frequency of the reflected wave may decrease. In other words, the sensor unit 130 may acquire sleep sensing information on breathing of the user by sensing movement of the user's body (e.g., the abdomen, the chest, etc.) based on a change in the phase difference or frequency of the reflected wave. For example, as shown in FIG. 3, the sensor unit 130 may be present at an area in an area in which the user goes to sleep. The sensor unit 130 may transmit the radio wave having the specific frequency through the transmission module and receive the reflected wave that is reflected by the user's body in response to the radio wave through the reception module. Also, the sensor unit 130 may acquire at least one of information on breathing, information on movement, and information on heart rate of the user based on the phase difference or frequency change of the reflected wave received through the reception module. However, a location at which the sensor unit of the present disclosure is provided is not limited to the location shown in FIG. 3, and a plurality of sensor units may be provided at various locations in an area in which the user goes to sleep.

Also, as described above, the sensor unit 130 may include the transmission module and the reception module to acquire sleep sensing data through a radio frequency (RF) sensing method, but the present disclosure is not limited thereto. In an additional embodiment, the sensor unit 130 of the present disclosure may further include a sensor module for transmitting and detecting WiFi radio waves, a sensor module for detecting airflow related to the user's respiration, etc. to acquire sleep sensing data.

According to an embodiment of the present disclosure, the sensor unit 130 may include one or more environment sensing module for acquiring indoor environment information including at least one piece of information on the user's body temperature, an indoor temperature, indoor air current, an indoor humidity, indoor sound, and indoor brightness with regard to a sleep environment of the user. The indoor environment information is information on the sleep environment of the user and may be information based on which influence of external factors on the user's sleep is taken into consideration through sleep states related to a change in the sleep stage of the user. The one or more environment sensing module may include, for example, at least one sensor module among a temperature sensor, an air current sensor, a humidity sensor, an acoustic sensor, and a brightness sensor. However, the one or more environment sensing module is not limited thereto and may further include various sensors that may have influence on the user's sleep.

According to an embodiment of the present disclosure, the computing device 100 may adjust the sleep environment of the user through the environment setup unit 140. Specifically, the environment setup unit 140 may include one or more driving modules, and adjust the sleep environment of the user by operating a driving module related to at least one of a temperature, a wind direction, a humidity, sound, and brightness based on an environment control signal received from the processor 170. The environment control signal may be a signal generated by the processor 170 based on a sleep state determination according to a change in the sleep stage of the user. For example, the environment control signal may be a signal for lowering the temperature, increasing the humidity, lowering the brightness, or lowering the sound with regard to the sleep environment of the user. The above description of the environment control signal is merely an example, and the present disclosure is not limited thereto.

The one or more driving modules may include, for example, at least one of a temperature control module, a wind direction control module, a humidity control module, an acoustic control module, and a brightness control module. However, the one or more driving modules are not limited thereto and may further include various driving modules that may bring changes to the sleep environment of the user. In other words, the environment setup unit 140 may adjust the sleep environment of the user by operating the one or more driving modules based on the environment control signal of the processor 170.

According to another embodiment of the present disclosure, the environment setup unit 140 may be implemented through Internet of things (IoT) connections. Specifically, the environment setup unit 140 may be implemented through connections with various devices that may bring changes to an indoor environment with regard to an area in which the user is present for a sleep. For example, the environment setup unit 140 may be implemented as a smart air conditioner, a smart heater, a smart boiler, a smart window, a smart humidifier, a smart dehumidifier, a smart light, etc. based on an IoT connection. The above-described details of the environment setup unit are merely examples, and the present disclosure is not limited thereto.

According to an embodiment of the present disclosure, the computing device 100 may include the transmission module 150 that transmits a wireless signal and the reception module 160 that receives the transmitted wireless signal. In an exemplary embodiment, the wireless signal may indicate an OFDM signal. For example, the wireless signal may be a WiFi-based OFDM sensing signal. Also, the transmission module 150 of the present disclosure may be implemented through a WiFi transmitter, and the reception module 160 may be implemented through a notebook computer, a smartphone, a tablet PC, etc. For example, the transmission module 150 and the reception module 160 may be equipped with a wireless chip conforming to WiFi 802.11n, 802.11ac, or another standard supporting OFDM. In other words, the computing device 100 may be implemented to acquire object state information with high reliability through relatively inexpensive equipment.

The transmission module 150 may transmit the wireless signal in one direction in which an object is present. The reception module 160 may be provided at the predetermined separation distance from the transmission module 150 and may receive the wireless signal transmitted by the transmission module 150. The wireless signal is an OFDM signal and thus may be transmitted or received through a plurality of subcarriers.

According to an embodiment, the transmission module 150 and the reception module 160 may transmit and receive an OFDM signal through one or more antennas. For example, when each of the transmission module 150 and the reception module 160 includes three antennas, channel state information on a total of 192 (i.e., 3×64) channels may be acquired at each frame through the three antennas and 64 subcarriers. The detailed numbers of antennas and subcarriers are merely examples, and the present disclosure is not limited thereto.

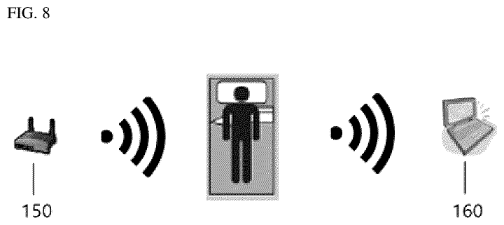

The transmission module 150 and the reception module 160 may be provided at the predetermined separation distance. In this case, the predetermined separation distance may mean an area in which an object is active or present. For example, the predetermined separation distance between the transmission module 150 and the reception module 160 may indicate an area in which the user goes to sleep. As a specific example, sleeping user may be present in an area between the transmission module 150 and the reception module 160 as shown in FIG. 8. In this case, the computing device 100 of the present disclosure may acquire object state information which is information on the user's movement or breathing based on a WiFi-based OFDM signal transmitted and received by the transmission module 150 and the reception module 160.

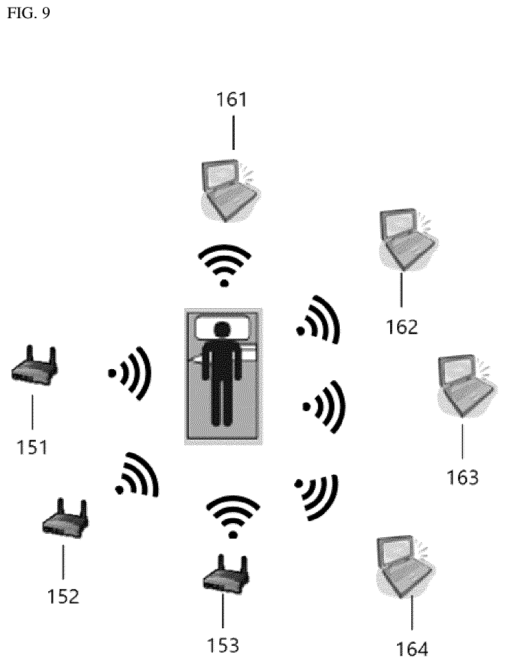

According to an embodiment, a plurality of transmission modules 150 and a plurality of reception modules 160 may be provided. As a specific example, each of three transmission modules 151, 152, and 153 and each of four reception modules 161, 162, 163, and 164 may be provided at the predetermined separation distance as shown in FIG. 9. In this case, wireless signals transmitted and received by the plurality of transmission modules and reception modules may differ from each other. However, locations at which the transmission modules and the reception modules of the present disclosure are provided and transmission and reception directions of wireless signals are not limited to those shown in FIG. 9.

According to an embodiment of the present disclosure, the processor 170 may have one or more cores and include a central processing unit (CPU), a general-purpose graphics processing unit (GPGPU), a tensor processing unit (TPU), etc. of the computing device for data analysis and deep learning.

The processor 170 may read the computer program stored in the memory 120 to perform data processing for machine learning according to the embodiment of the present disclosure. According to an embodiment of the present disclosure, the processor 170 may perform computation for training a neural network. The processor 170 may perform computation for training a neural network such as processing of input data for deep learning, extraction of features from input data, error calculation, update of weights of the neural network through backpropagation, etc.

Also, at least one of the CPU, the GPGPU, and the TPU of the processor 170 may process training of a network function. For example, the CPU and the GPGPU may process training of a network function and data classification through the network function together. Also, in an embodiment of the present disclosure, processors of a plurality of computing devices may be used together to process training of a network function and data classification through the network function. The computer program executed in the computing device according to an embodiment of the present disclosure may be a program executable by the CPU, the GPGPU, or the TPU.

In the present specification, a network function may be interchangeably used with an artificial neural network or neural network. In the present specification, a network function may include one or more neural networks, and in this case, an output of the network function may be an ensemble of outputs of the one or more neural networks.

In the present specification, a model may include a network function. A model may include one or more network functions, and in this case, an output of the model may be an ensemble of outputs of the one or more network functions.

The processor 170 may read the computer program stored in the memory 120 and provide a sleep assessment model and a sleep analysis model according to an embodiment of the present disclosure. According to an embodiment of the present disclosure, the processor 170 may perform calculation to generate sleep analysis information based on sleep sensing data. According to an embodiment of the present disclosure, the processor 170 may perform calculation for training the sleep assessment model and the sleep analysis model.

According to an embodiment of the present disclosure, the processor 170 may generally process overall operations of the computing device 100. The processor 170 may provide or process appropriate information or functions to the user terminal by processing signals, data, information, etc. input or output through the above-described components or executing the application program stored in the memory 120.

A method of predicting a sleep state based on data measured in a sleep environment of a user will be described in detail below.

According to an embodiment of the present disclosure, the processor 170 may acquire sleep sensing data of a user. Acquiring sleep sensing data according to an embodiment of the present disclosure may be receiving or loading sleep sensing data stored in the memory 120. Acquiring sleep sensing data may be receiving or loading sleep sensing data from another computing device or a separate processing module in the same computing device to another storage medium based on a wired/wireless communication means.

The processor 170 may receive sleep sensing data related to a sleep environment of the user. According to an embodiment, the sleep sensing data may include information on breathing of the user during a predetermined time period with regard to the sleep environment of the user. For example, the sleep sensing data may be information on breathing of the user acquired for sixty minutes. The sleep sensing data may be information on breathing of the user measured at 10 Hz for sixty minutes and may be time-series information having a matrix size of 3,600×10. The specific values of the sleep sensing data are merely examples, and the present disclosure is not limited thereto. The sleep sensing data may be input data that is processed as an input to a sleep assessment model which is a neural network model to predict a result value (i.e., sleep analysis information) through the sleep assessment model.

In an additional embodiment, the sleep sensing data may further include information on movement and information on heart rate of the user acquired through the sensor unit 130 during a predetermined time period with regard to the sleep environment of the user. In other words, according to an implementation form of the sensor unit 130, the sleep sensing data may include at least one of information on breathing, information on movement, and information on heart rate of the user. In this case, the sleep sensing data may include information on breathing of the user measured at 10 Hz for sixty minutes and information on movement and information on heart rate of the user measured at 1 Hz for sixty minutes. The above specific values related to sleep sensing data are merely examples, and the present disclosure is not limited thereto.

According to an embodiment of the present disclosure, sleep analysis information predicted by the sleep assessment model based on the sleep sensing data may show high accuracy when three elements (i.e., information on breathing, information on movement, and information on heart rate) are acquired through the sensor unit 130. However, the three elements (i.e., information on breathing, information on movement, and information on heart rate) acquired through the sensor unit 130 are not necessarily required as an input to the sleep assessment model for predicting sleep analysis information in the present disclosure.

According to an embodiment of the present disclosure, the processor 170 may predict sleep analysis information. Specifically, the processor 170 may input the sleep sensing data of the user to the sleep assessment model to predict sleep analysis information on the user. Here, the sleep assessment model is a model for predicting a sleep state of a user and predicting a probability of the user developing a disorder and may provide sleep analysis information based on data acquired from a user's sleep.

As a model for predicting information on at least one of a sleep state of the user and a probability of the user developing a disorder, the sleep assessment model may include one or more network functions.

More specifically, the processor 170 may generate training datasets for training the sleep assessment model. The processor 170 may receive sleep diagnosis datasets including a plurality of pieces of sleep diagnosis data each corresponding to a plurality of users. The sleep diagnosis data may be information received from the external server 20. The sleep diagnosis data may include information on a sleep checkup of a specific user and may be, for example, information on polysomnography. Information on polysomnography of a user may include information on an electroencephalogram (EEG), an electrooculogram (EOG), a jaw electromyogram (EMG), respiration, an electrocardiogram, an arterial blood oxygen saturation level, a tibialis anterior EMG, and other physiological and physical parameters over several hours.

Also, the information on the polysomnography may include information on sleep analysis results obtained through the above information. As a specific example, information that an apnea-hypopnea index (AHI) related to a sleep apnea index is 21 and information that a sleep stage of the user at a specific time point is REM sleep may be included as diagnosis results based on information acquired during a sleep period of the user. An AHI is information that is a criterion for diagnosing sleep apnea. In general, when an AHI is measured to be less than 5, this case is classified as normal. When an AHI is measured to be 5 or more and less than 15, this case is classified as mild sleep apnea. When an AHI is measured to be 15 or more and less than 30, this case is classified as moderate sleep apnea. When an AHI is measured to be 30 or more, this case is classified as severe sleep apnea.

The processor 170 may generate a training input dataset by extracting information on the user's respiration, heart rate, and movement during a predetermined time period from each of the plurality of pieces of sleep diagnosis data. For example, the processor 170 may extract information on breathing of the user during one hour from the sleep diagnosis data. In this case, training input data may be the user's information on breathing measured at 10 Hz for sixty minutes and may be time-series information having a matrix size of 3,600×10.

In other words, the training input dataset may include at least one type of information among the pieces of information on the respiration, the heart rate, and the movement during the predetermined time period extracted from each of the plurality of pieces of sleep diagnosis data.

Also, the processor 170 may extract at least one of information on apnea indices, information on sleep states, and information about whether a disorder has occurred from each of the plurality of pieces of sleep diagnosis data to generate training output data.

As a specific example, the processor 170 may extract information on an apnea index (e.g., an AHI of 19) of user A from sleep diagnosis data of user A. As another example, the processor 170 may extract information that user B has a heart disease related to irregular pulses from sleep diagnosis data of user B. As still another example, the processor 170 may extract information on sleep stages of user C corresponding to different time points from sleep diagnosis data of user C. The above-described details of training output data are examples, and the present disclosure is not limited thereto.

That is, the processor 170 may generate a training output dataset based on each of the plurality of pieces of sleep diagnosis data. In other words, a training output dataset may be information on a sleep measurement result (or a sleep diagnosis result) extracted from each of the plurality of pieces of diagnosis data.

Also, the processor 170 may generate a labeled training dataset by matching the training input dataset with the training output dataset. For example, when first training input data is generated by extracting information on breathing measured during a predetermined time period (e.g., time-series data related to breathing measured for one hour) from sleep diagnosis data of a first user and first training output data is generated by extracting information that a sleep diagnosis result corresponding to the information measured during the predetermined time period is an AHI of 19, the processor 170 may generate a labeled training dataset by matching the first training input data and the first training output data, which are acquired from the same user, the first user, at the same time, with each other. In other words, the processor 170 may generate a labeled training dataset by matching each piece of training input data with a corresponding piece of training output data.

According to an embodiment of the present disclosure, the processor 170 may train the sleep assessment model. Specifically, the processor 170 may train the one or more network functions constituting the sleep assessment model using labeled training datasets. Specifically, the processor 170 may input each of training input datasets into the one or more network functions and compare each of output data predicted by the one or more network functions with a training output dataset corresponding to a label of the training input dataset, thereby deriving an error. In other words, in training of a neural network, training input data may be input to input layers of one or more network functions, and training output data may be compared with outputs of the one or more network functions. The processor 170 may train a neural network based on errors between calculation results of the one or more network functions and training output data (label) with respect to the training input data.

Also, the processor 170 may adjust weights of the one or more network functions based on the errors using a backpropagation method. In other words, the processor 170 may adjust the weights based on the errors between the calculation results of the one or more network functions and the training output data with respect to the training input data so that the outputs of the one or more network functions may approximate to the training output data.

When training of the one or more network functions is performed for predetermined epochs or more, the processor 170 may determine whether to stop the training using verification data. The predetermined epochs may be a part of total training-target epochs. The verification data may be at least a part of the labeled training datasets. In other words, the processor 170 may train the neural network using the training datasets and, after training of the neural network is repeated for the predetermined epoch or more, may determine whether the effect of training the neural network is a predetermined level or above using verification data. For example, in the case of performing training to be repeated ten times using one hundred pieces of training data, the processor 170 may repeat training ten times which are predetermined epochs and then repeat training three times using ten pieces of verification data. When a change in the output of the neural network during the three times of repeated training is the predetermined level or less, the processor 170 may determine that further training is meaningless and terminate the training. In other words, the verification data may be used for determining the end of training based on whether epoch-specific training effects are at the certain level or above in repeated training of the neural network. The above numbers of pieces of training data, verification data and the above number of repetitions are merely examples, and the present disclosure is not limited thereto.

The processor 170 may test performance of the one or more network functions using test datasets and determine whether to activate the one or more network functions, thereby generating a sleep assessment model. Test data may be used for verifying the performance of the neural network and may be at least a part of the training datasets. For example, 70% of the training datasets may be used for training the neural network (i.e., training for adjusting weights so that a result value similar to the label may be output), and 30% may be used as test data for verifying performance of the neural network. The processor 170 may input test datasets to the neural network having completed learning, measure errors, and determine whether to activate the neural network according to whether the performance is predetermined performance or above. The processor 170 may verify performance of the neural network having completed learning using the test data on the neural network having completed learning and activate the neural network so that the neural network may be used by another application when the performance of the neural network having completed learning is the predetermined reference or above. Also, when the performance of the neural network having completed learning is the predetermined reference or below, the processor 170 may deactivate and discard the neural network. For example, the processor 170 may determine performance of a generated neural network model based on elements such as accuracy, precision, recall, etc. The above-described performance assessment elements are merely examples, and the present disclosure is not limited thereto. According to an embodiment of the present disclosure, the processor 170 may generate a plurality of neural network models by independently training neural networks, assess performance of the neural network models, and use only neural networks showing a certain performance level or above to predict sleep analysis information.

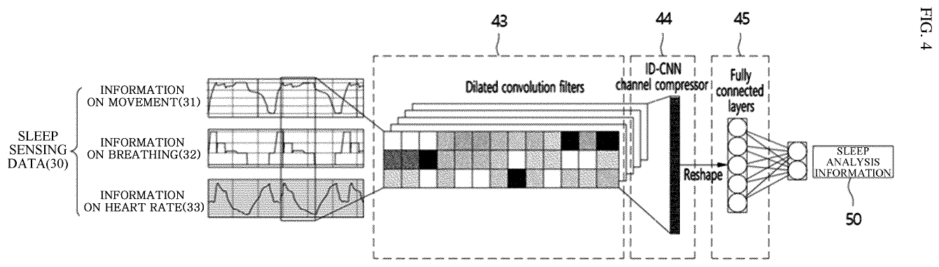

A data computation process of one or more network functions (or neural networks) will be described in further detail below with reference to FIG. 4.

Sleep sensing data 30 used as an input to a neural network in the present disclosure may include information on breathing 32 of a user acquired during a predetermined time period with regard to a sleep environment of the user. For example, the sleep sensing data 30 may be the information on breathing 32 of the user acquired for sixty minutes. In this case, the sleep sensing data 30 may be the information on breathing 32 of the user measured at 10 Hz for sixty minutes and may be time-series information having a matrix size of 3,600 (60 minutes×60 seconds)×10. In other words, data used as an input to a neural network in the present disclosure may be relatively long data. When data used as an input to a neural network is long time-series data, it may be difficult for the neural network to perform calculation.

For example, in the case of a general convolutional neural network (CNN), a receptive field may be expanded by increasing a kernel size or maximizing a depth (the number of filters). The receptive field may be related to the domain of a filter for processing data related to an input to the neural network. For example, when the receptive field expands, the amount of information used in a process of predicting an output may increase. When the receptive field shrinks, the amount of information used in a process of predicting an output may decrease.

However, as described above, when the kernel size or the depth is increased, the number of parameters increases, and thus there is a risk of overfitting. Also, in a computation process of a general CNN, the length of an output end corresponding to an input may be reduced according to a stride related to an interval between locations to which a filter is applied. For example, when the stride is 2, intervals of a filter are 2, and thus an output length may be half an input length. Also, data loss may occur in a pooling process of a general CNN. For example, in the case of max pooling, only a maximum record is extracted as a feature from among records included in a specific kernel size, and thus feature values corresponding to other records may be lost. For time-series data, locations of input points should be maintained, but each data lengths of an input end and an output end may vary in a computation process.

In other words, in the case of predicting sleep environment sensing data (i.e., long time-series data) related to an input to a neural network of the present disclosure through a general CNN, there is a risk of overfitting with an increase in the number of parameters, and it may be difficult to learn a temporal relationship of time-series data due to a length change of an input and an output.

In an additional embodiment, the sleep sensing data 30 may further include information on movement 31 and information on heart rate 33 of the user during the predetermined time period. In this case, the sleep sensing data includes the information on breathing 32 of the user measured at 10 Hz for sixty minutes and the information on movement 31 and the information on heart rate 33 of the user measured at 1 Hz for sixty minutes and thus may be time-series information having a matrix size of (3,600×10)+(3,600×1)+(3,600×1). In this case, data much longer than sleep sensing data including only the information on breathing 32 is used as an input to a neural network, and thus efficiency in calculation and learning through a CNN may be further degraded.

Accordingly, the one or more network functions constituting the sleep assessment model of the present disclosure may include a dilated CNN 43 for strengthening a long-term relationship without loss of input data. The dilated CNN 43 strengthens a long-term relationship by expanding a receptive field of a filter related to an input to the one or more network functions and maintains lengths of inputs and outputs of the one or more network functions by adding zero-padding to an input-related filter. Specifically, the dilated CNN may expand the receptive field by considering a dilation rate, which is the distance between kernels, as a parameter. For example, a 3×3 kernel having a dilation rate of 2 may have the same field of view as a 5×5 kernel, and thus the receptive field may be expanded. In other words, while the number of parameters is maintained, the receptive field is expanded to strengthen a long-term relationship such that long-time series data can be processed. Also, the dilated CNN 43 can prevent loss by adding zero-padding to lost records (i.e., skipped records) which may occur in a dilation operation process. Accordingly, it is possible to reduce loss of features and also maintain lengths of inputs and outputs (maintain temporal correlations).

Also, the one or more network functions may include a one-dimensional (1D)-CNN 44. The 1D-CNN 44 may be a network function for reducing dimensions of long time-series data. The 1D-CNN 44 may be a network function located at the second half of the neural network to reduce (or compress) dimensions of data transferred from the dilated CNN 43.

In other words, input data (e.g., sleep environment sensing data and training input data) input to an input layer may be passed through a computation process of the dilated CNN 43, reduced in dimensionality through the 1D-CNN 44, and output by a fully connected layer (FCL) 45.

The sleep assessment model of the present disclosure expands a receptive field through a dilated CNN while maintaining the number of parameters required for computation. Accordingly, the sleep assessment model can strengthen a long-term relationship, prevent loss of features which occurs in the computation process, and maintain lengths of inputs and outputs (maintain temporal correlations). Therefore, the processor 170 can generate a sleep assessment model for predicting sleep analysis information 50 based on sleep sensing data by training a neural network model including a dilated CNN using training datasets.

The sleep assessment model may output the sleep analysis information 50 using sleep-related sensing data as an input. The sleep analysis information 50 may include at least one of apnea severity information on a degree of apnea occurrence during the user's sleep, sleep stage information on changes in the sleep state, and disorder prediction information on a probability of disorder occurrence.

The apnea severity information may include information on AHI measured during a predetermined time period with regard to the sleep environment of the user. For example, the apnea severity information may include information that an AHI for ninety minutes is 8, and the user may be determined to correspond to mild sleep apnea based on the index. The sleep stage information may include changes in one or more sleep states over time with regard to the sleep environment of the user. For example, the sleep stage information may include information on a sleep stage of the user at each time point during a predetermined time period or information on ratios of sleep stages. The disorder prediction information may include prediction information on at least one of sleep disorders, mental disorders, brain disorders, and cardiovascular disorders. For example, the disorder prediction information may include information that a probability of the user developing a brain disorder in three years is 40%. The above-described details of the apnea severity information, the sleep stage information, and the disorder prediction information are merely examples, and the present disclosure is not limited thereto.

That is, the sleep assessment model of the present disclosure may be a raw-data-to-output (or end-to-end deep learning) model that outputs output data (i.e., sleep analysis information) without a preprocessing operation for input data (sleep sensing data) which is time-series information. In other words, a data preprocessing operation may be skipped in a data computation process employing a neural network.

Also, the sleep sensing data which is processed as an input to the sleep assessment model may include information on breathing of the user measured during a predetermined time period. In other words, the sleep assessment model of the present disclosure may be a neural network model that predicts the above-described sleep analysis information based on minimal elements (i.e., information on breathing of the user). In general, various information (e.g., brain waves, an EOG, a jaw EMG, respiration, an electrocardiogram, an arterial blood oxygen saturation level, a tibialis anterior EMG, and other physiological and physical variables) may be required for sleep assessment (or analysis). The present disclosure is to provide a user with convenience in acquiring sleep assessment information and may provide a neural network model (i.e., a sleep assessment model) that uses only respiration-related information during the user's sleep as input data. Accordingly, a sensing process for acquiring a plurality of pieces of input data may be scaled down. This facilitates assessment (or analysis) of a user's sleep in real life and thus can provide convenience in continuous healthcare.

In an additional embodiment, the sleep sensing data may further include information on movement and information on heart rate of the user measured during a predetermined time period. In this case, the sleep assessment model may be a neural network model that is trained to provide sleep analysis information based on three elements (i.e., information on breathing, information on movement, and information on heart rate of a user). In this case, the sleep sensing data includes the information on breathing, the information on movement, and the information on heart rate of the user, and thus input data to a neural network may include the three elements. In other words, since the three elements are taken into consideration as input data, accuracy in sleep analysis information predicted by the sleep assessment model can be further improved.

According to an embodiment of the present disclosure, the processor 170 may generate healthcare information for improving the sleep environment and health of the user based on the sleep analysis information. The healthcare information may include at least one of information on eating habits, information on the amount of exercise, and information on an optimal sleep environment.

As a specific example, when the sleep analysis information includes information that the AHI is predicted to be 13 which corresponds to mild sleep apnea, the processor 170 may generate healthcare information including “information for recommending weight loss through exercise,” “information for avoiding drugs relaxing muscles,” etc.

As another example, when the sleep analysis information includes information that the AHI is predicted to be 32 which corresponds to severe sleep apnea, the processor 170 may generate healthcare information including “information for recommending treatment with a continuous positive airway pressure (CPAP) machine.”

As still another example, when the sleep analysis information includes information that a probability of developing arrhythmia in three years is 70%, the processor 170 may generate healthcare information including “information on eating habits for recommending using vegetable oil, such as soy bean oil, perilla oil, and sesame oil, rather than animal fat and eating food high in cholesterol, such as eggs, fish, etc., two or three times or less a week,” “information on the amount of exercise for avoiding excessive strength training and recommending thirty minutes of aerobic exercise a day,” etc. The above-described details of information included in sleep analysis information and healthcare information are merely examples, and the present disclosure is not limited thereto.

Also, the processor 170 may determine to transmit the healthcare information generated based on the sleep analysis information to the user terminal 10. Accordingly, the user can easily recognize information for improving apnea occurring in his or her sleep or lowering a probability of developing an associated disorder.

According to an embodiment of the present disclosure, the processor 170 may derive a correlation between the sleep sensing data and the disorder prediction information based on a variation of the disorder prediction information output by changing the information on breathing for the predetermined time period which is included in the sleep sensing data of the user input to the sleep assessment model.

For example, the processor 170 may adjust the information on breathing for the predetermined time period included in the sleep sensing data related to the sleep environment of the user. In this case, with a change of the input to the neural network model (i.e., the sleep assessment model), the disorder prediction information may be changed and output. In other words, the processor 170 may derive a correlation between the input to the neural network and the sleep analysis information which is changed and output according to the sleep sensing data. For example, the correlation between the sleep sensing data and the disorder prediction information may be information that a probability of developing a cardiovascular disorder increases with a decrease in the period of the user's apnea during his or her sleep (i.e., even with the same apnea index, a probability of developing a disorder may vary depending on the period of occurrence). The above-described details of the correlation between the sleep sensing data and the disorder prediction information are merely examples, and the present disclosure is not limited thereto.

In other words, the correlation between information on breathing and disorder prediction information rather than the generally known correlation between breathing during a sleep and a disorder predicted according to the breathing is derived through a neural network such that a meaningful insight on the relationship between breathing and a disorder can be provided.

According to an embodiment of the present disclosure, the processor 170 may generate sleep degradation factor information based on sleep stage information and indoor environment information. The indoor environment information is information on the sleep environment of the user acquired by the sensor unit 130 and may include information on at least one of the body temperature of the user, the indoor temperature of a room in which the user is present, an indoor humidity, an indoor sound, and an indoor brightness.

The sleep degradation factor information may be information on an external factor that degrades the quality of the user's sleep during the user's sleep process. For example, the sleep degradation factor information may include information that the quality of the user's sleep is degraded with a decrease in the indoor temperature at a specific time point. As another example, the sleep degradation factor information may include information that the quality of the user's sleep is degraded with an increase in the brightness at a specific time point. The above-described details of sleep degradation factor information are merely examples, and the present disclosure is not limited thereto.

More specifically, the processor 170 may identify a first time point at which the quality of the user's sleep is degraded based on the sleep stage information. In general, sleep stages may be classified into one or more stages by the degree of sound sleep of a user. For example, sleep stages may be classified into a first stage to a fourth stage, and a higher stage represents that a user is in a deeper sleep. These sleep stages may be repeated several times based on a certain time period (e.g., one and half hour) during a user's daily sleep. In this case, the processor 170 may identify the first time point at which the quality of the user's sleep is degraded through the sleep stage information included in the sleep analysis information predicted by the sleep assessment model. As a specific example, when the sleep stage of the user changes from the third stage to the first stage at 3:10 a.m. (i.e., a deep sleep changes to a light sleep regardless of repeated cycles of a sleep), the processor 170 may identify the corresponding time point (i.e., 3:10) as a first time point at which the quality of the user's sleep is degraded. The above-described detail of the first time point is merely an example, and the present disclosure is not limited thereto.

Also, the processor 170 may identify a singularity related to a variation of the indoor environment information corresponding to the first time point. The indoor environment information is information on the environment of an area in which the user goes to sleep. The indoor environment information may include at least one piece of information on the user's body temperature, an indoor temperature, indoor air current, an indoor humidity, indoor sound, and indoor brightness and may be acquired through the sensor unit 130.

More specifically, the processor 170 may identify a singularity related to a variation of the indoor environment information based on whether a variation of one or more pieces of information included in the indoor environment information exceeds a predetermined threshold variation corresponding to the first time point.

As a specific example, when the first time point at which the quality of the user's sleep is degraded is 2:40 a.m., the processor 170 may identify a variation of the one or more pieces of information included in the indoor environment information corresponding to the time point (i.e., 2:40). In this case, a temperature variation may be identified as +3° C. When the predetermined threshold variation of the temperature is +2° C., the processor 170 may identify that the indoor temperature is a singularity related to volatility of indoor environment information. In other words, the processor 170 may identify temperature as an element that degraded the quality of the user's sleep during the sleep. Also, the processor 170 may generate sleep degradation factor information based on the identified singularity. For example, the processor 170 may generate sleep degradation factor information that the quality of the user's sleep was degraded due to the change in the indoor temperature at 2:40. In other words, the sleep degradation factor information generated by the processor 170 may include information on the time point at which the quality of the user's sleep was degraded and external factor information on the sleep quality degradation. Further, for example, the foregoing temperature change may have no influence on a change in the sleep stage of user A (i.e., does not degrade the sleep quality of user A) but may have influence on a change in the sleep stage of user B (i.e., degrades the sleep quality of user B). In other words, sleep degradation factor information may reflect a sleep-related characteristic of each individual. The above-described details of the first time point, the variation of indoor environment information, and the predetermined threshold variation are merely examples, and the present disclosure is not limited thereto.

According to an embodiment of the present disclosure, the processor 170 may generate an environment control signal for controlling the environment setup unit 140 based on the sleep degradation factor information. For example, when the sleep degradation factor information includes information that the quality of the user's sleep was degraded by a change in the indoor temperature at 2:40, the processor 170 may generate an environment control signal for causing the environment setup unit 140 to adjust the temperature at the time point based on the sleep degradation factor information. In other words, the processor 170 may generate an environment control signal for controlling the environment setup unit 140 based on the specific time point and the sleep degradation factor included in the sleep degradation factor information to change the indoor environment of an area in which the user goes to sleep. Accordingly, the environment setup unit 140 can operate the one or more driving modules based on the environment control signal to adjust the indoor environment in which the user is present. In this case, since sleep degradation factor information is generated based on a change in the sleep stage of each individual and an environment control signal is information generated based on sleep degradation factor information such as that described above, a sleep environment adjustment operation in the present disclosure may reflect a characteristic of each individual user related to a sleep environment.

In other words, the processor 170 generates an environment control signal for operating the one or more driving modules included in the environment setup unit 140 based on sleep degradation factor information and thus can provide an optimal sleep environment for each of a plurality of users. Accordingly, users' sleep efficiency can be improved.

A method of providing sleep-related analysis information will be described in detail below.

According to an embodiment, sleep sensing data may be a bio-signal acquired during a user's sleep. For example, the sleep sensing data may include a bio-signal measured from the user with regard to polysomnography. Bio-signals related to polysomnography may include information on brain waves, an EOG, a jaw EMG, respiration, an electrocardiogram, an arterial blood oxygen saturation level, a tibialis anterior EMG, and other physiological and physical variables. According to an additional embodiment, bio-signals related to polysomnography may further include information on breathing and movement during a user's sleep. The sleep sensing data may be acquired through one or more channels. Each of the one or more channels may be related to each pair of electrodes configured to acquire a bio-signal.

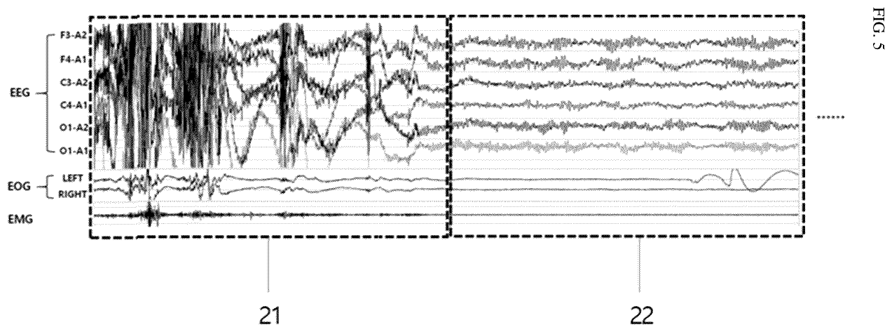

For example, the sleep sensing data may be a bio-signal measured with regard to polysomnography and may include information on an EEG, an EOG, and an EMG. In this case, the information on the EEG, the EOG, and the EMG may be acquired through different channels. As a specific example, as shown in FIG. 5, the information on the EEG may be acquired through six channels (i.e., F3-A2, F4-A1, C3-A2, C4-A1, O1-A2, and O1-A1), the information on the EOG may be acquired through two channels (LEFT and RIGHT), and the information on the EMG may be acquired through one channel. In other words, sleep sensing data of the present disclosure means bio-signals related to an EEG, an EOG, and an EMG measured for polysomnography from a user's body during a sleep period. The bio-signals may be acquired through the six channels related to an EEG, the two channels related to an EOG, and the one channel related to an EMG, that is, nine channels in total. As described above, sleep sensing data based on which sleep analysis information is generated includes various bio-signals acquired through a plurality of channels, and thus output accuracy of an artificial neural network can be improved.

Also, sleep sensing data of the present disclosure may be time-series data acquired during a sleep period of a user. In this case, the sleep sensing data may be a combination of one or more pieces of sequential data that are divided by predetermined unit time. The predetermined unit time may be, for example, thirty seconds which are a reference for detecting a change in sleep. In other words, the sleep sensing data may include one or more pieces of sequential data related to bio-signals of the user acquired in time series through one or more channels with regard to a sleep environment of the user. For example, the one or more pieces of sequential data may include information on an EEG, an EOG, and an EMG acquired through the nine channels based on the predetermined unit time (e.g., thirty seconds).

As a specific example, referring to FIG. 5, one or more pieces of sequential data included in sleep sensing data may include first sequential data 21 and second sequential data 22. The first sequential data 21 may be bio-signals related to an EEG, an EOG, and an EMG acquired through the nine channels from zero seconds to thirty seconds. The second sequential data 22 may be bio-signals related to an EEG, an EOG, and an EMG acquired through the nine channels from thirty-one seconds to sixty seconds which are time points subsequent to the first sequential data 21. The above description of FIG. 5 is merely an example for helping understanding of the present disclosure, and one or more pieces of sequential data included in sleep sensing data of the present disclosure are not limited to first and second sequential data. In other words, sleep sensing data of the present disclosure is data related to bio-signals measured during a sleep period (e.g., eight hours) of a user, and thus it is apparent to those of ordinary skill in the art that sleep sensing data may include a larger number of pieces of sequential data.

According to an embodiment of the present disclosure, the processor 170 may preprocess the sleep sensing data. The preprocessing of the sleep sensing data according to an embodiment may include downsampling of the sleep sensing data. For example, sleep sensing data acquired through multiple channels (e.g., nine channels) for polysomnography may be acquired at different sampling rates but may have a sampling rate that is unnecessarily high for measuring a sleep stage. Accordingly, the processor 170 may downsample data acquired through each channel at an appropriate sampling rate for a neural network model of the present disclosure. As a specific example, data acquired through each channel may be downsampled at a sampling rate of 25 Hz to increase the period of each signal. This can prevent aliasing by reducing distortion of data acquired in time series. Accordingly, it is possible to clearly distinguish between pieces of data acquired through channels.

In an additional embodiment, the preprocessing of the sleep sensing data may include preprocessing for removing noise from data acquired through each channel. Specifically, the processor 170 may standardize the magnitude of a signal included in each piece of data acquired through each channel based on the comparison between the magnitude of the signal and a predetermined reference signal magnitude. For example, when the magnitude of a signal included in each piece of sleep sensing data acquired through a plurality of channels is less than the reference signal magnitude, the processor 170 may adjust the magnitude of the signal to be larger. Also, when the magnitude of a signal included in each piece of sleep sensing data acquired through each channel is the reference signal magnitude or more, the processor 170 may adjust the magnitude of the signal to be smaller (i.e., not to be clipped). The above-described details of preprocessing of sleep sensing data are merely example, and the present disclosure is not limited thereto.

According an embodiment of the present disclosure, the processor 170 may output sleep analysis information by processing the sleep sensing data as an input to the sleep analysis model. The sleep analysis information may include sleep stage information indicating that the user's sleep corresponds to at least one of one or more sleep stages. In other words, the sleep analysis information may include prediction information on a sleep stage during the user's sleep. For example, the sleep analysis information may include sleep stage information indicating that the user's sleep corresponds to at least one of the one or more sleep stages with regard to a specific time point. The one or more sleep stages may include, for example, wake (a non-sleep state), N1 (Non-REM 1), N2 (Non-REM 2), N3 (Non-REM 3), and REM sleep. As a specific example, the sleep analysis information may include information that the user's sleep stage at a first time point corresponds to REM sleep. The above-described detail of sleep analysis information is merely an example, and the present disclosure is not limited thereto.

According to an embodiment of the present disclosure, the processor 170 may train one or more network functions using training datasets. To this end, the processor 170 may receive training datasets from the external server 20. The external server 20 may be a server related to at least one of a hospital server and a governmental server and may receive checkup data of each of a plurality of users related to polysomnography from at least one of electronic health records, electronic medical records, and a health checkup DB of each server. The processor 170 may generate training datasets including a plurality of pieces of training data based on the checkup data of each of the plurality of users received from the external server 20. The training datasets may include training input datasets related to inputs to a neural network and training output datasets compared with outputs.

More specifically, with regard to a sleep environment of each of the plurality of users, the training datasets may include training input datasets related to bio-signals measured from the users' bodies through polysomnography and training output datasets related to sleep stage determination information corresponding to time points of the bio-signals. The processor 170 may generate a sleep analysis model of the present disclosure by training the one or more network functions using the training datasets.

The sleep analysis model may include a first sub-model 310 and a second sub-model 320. The first sub-model 310 and the second sub-model 330 are described below as, but not limited to, a dimension reduction sub-model 310 and a dimension recovery sub-model 330, respectively. The processor 170 may train the sleep analysis model using the training input data as an input to the dimension reduction sub-model 310 so that the dimension recovery sub-model 330 may output training data related to a label of the training input data.

The dimension reduction sub-model 310 may be a model that extracts a feature (i.e., embedding) using training input data related to bio-signals acquired in time series during a user's sleep. In other words, the dimension reduction sub-model 310 may receive training input data from the processor 170 and designate a feature vector column of the training input data as an output to learn an intermediate process in which the input data is converted into a feature.

Also, the processor 170 may transfer embedding (i.e., a feature) related to an output of the dimension reduction sub-model 310 to the dimension recovery sub-model 330. The dimension reduction sub-model 310 may output sleep analysis information on the feature using the feature as an input. The processor 170 may derive an error by comparing the sleep analysis information, which is an output of the dimension recovery sub-model, with training output data and adjust weights of each model based on the derived error using a backpropagation method.

The processor 170 may adjust weights of the one or more network functions based on the error between the calculation result of the dimension recovery sub-model 330 with respect to the training input data and the training output data so that the sleep analysis information which is the output of the dimension recovery sub-model 330 may approximate the training output data.

In other words, the dimension reduction sub-model may be trained to extract a feature from the training input data, and the dimension recovery sub-model may be trained to output analysis information corresponding to the extracted feature.

Also, the sleep analysis model may further include one or more attention modules. The one or more attention modules may predict attention weights for analysis information corresponding to a feature. Specifically, the processor 170 may cause the one or more attention modules to learn a matching relationship between an input (i.e., training input data) and an output (i.e., training output data) using the training input data and the training output data. Accordingly, the one or more attention modules 320 may generate association information on the association between a feature and a time step of the dimension recovery sub-model 330. In other words, in a neural network training process, the one or more attention modules may emphasize an element to be focused on between input data and output data by giving an attention weight to each element of the time-series input data. That is, the one or more attention modules may be modules trained to give a weight related to an association between an output value and an input value of the sleep analysis model.

As a specific example, the dimension recovery sub-model 330 may include one or more recurrent neural networks (RNNs) and may be a model that outputs prediction information on a second timestamp using prediction information on a first timestamp as an input. In this case, the first timestamp may be an earlier time point than the second timestamp. Specifically, prediction information predicted through a first RNN of the dimension recovery sub-model may be transferred to a second RNN of the next timestamp, and prediction information predicted through the second RNN may be transferred to a third RNN of the next timestamp. In this process, the one or more attention modules may determine a time point of a feature to be focused on through association information between time steps. In other words, the dimension recovery sub-model 330 determines a time point of a change factor to be focused on through the one or more attention modules in a process of repeatedly predicting prediction information through the one or more RNNs and thus may be trained to output prediction information on associations between time points of various factors.

In other words, the processor 170 may input the training input data to the dimension reduction sub-model so that a feature corresponding to the training input data may be output, and may process the feature as an input to the dimension recovery sub-model 330 through the one or more attention modules. In this case, the dimension recovery sub-model 330 may output sleep analysis information using the feature as an input, and the processor 170 may derive an error by comparing the output sleep analysis information with the training output data which is a label of the training input data and adjust weights of each model based on the derived error. Through the above-described training process, the processor 170 may train the one or more network functions to generate the sleep analysis model.

Accordingly, the sleep analysis model may output sleep analysis information using sleep sensing data as an input.

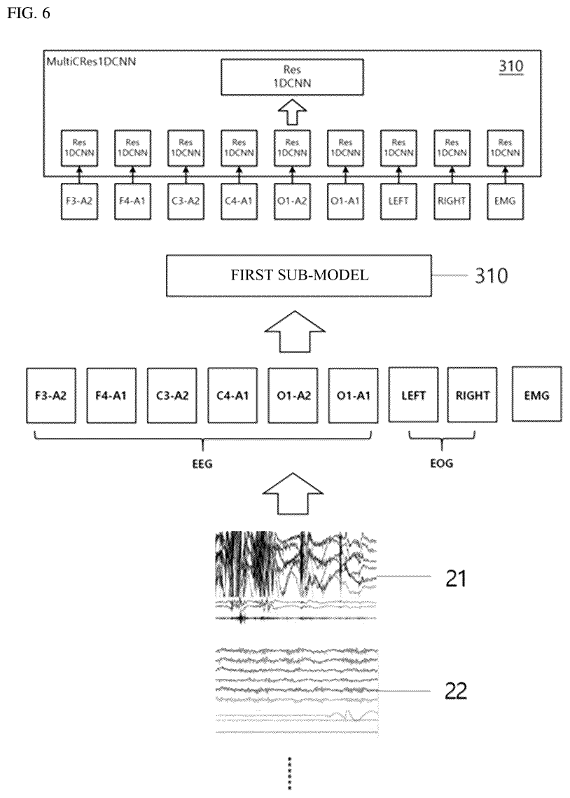

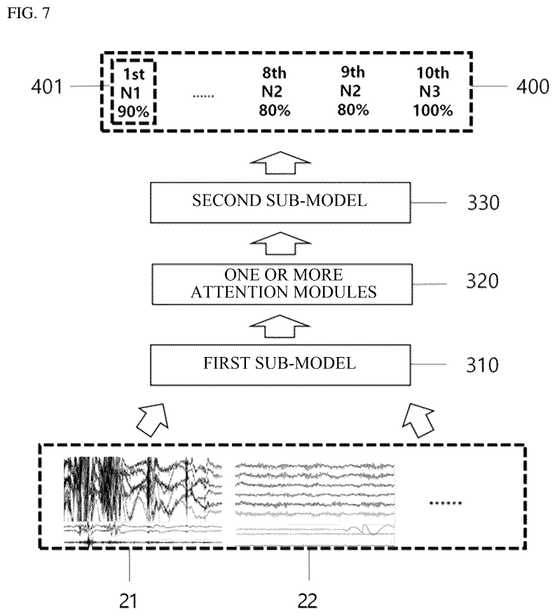

More specifically, the sleep analysis model may output sleep analysis information corresponding to each of one or more pieces of sequential data using sleep sensing data including the one or more pieces of sequential data as an input. In other words, the sleep analysis model may output sleep analysis information corresponding to each of one or more pieces of sequential data using the sleep sensing data as an input. The sleep analysis model may include a dimension reduction sub-model (e.g., an encoder) and a dimension recovery sub-model (e.g., a decoder). The dimension reduction sub-model and the dimension recovery sub-model described below may mean models trained through the above-described training process. A process in which the sleep analysis model outputs sleep analysis information corresponding to each piece of sequential data using sleep sensing data including one or more pieces of sequential data as an input will be described in detail below with reference to FIGS. 6 and 7.

The dimension reduction sub-model 310 may extract one or more features corresponding to each of one or more channels using one or more pieces of sequential data included in sleep sensing data as an input and generate one or more integrated features corresponding to each piece of sequential data by integrating the extracted one or more features.

More specifically, referring to FIG. 6, training input sleep sensing data of the present disclosure may be related to bio-signals of a user acquired in time series through one or more channels during the user's sleep. The sleep sensing data may include one or more pieces of sequential data. The one or more pieces of sequential data may include the first sequential data 21 and the second sequential data 22. The first sequential data 21 may be bio-signals related to an EEG, an EOG, and an EMG acquired through the nine channels from zero seconds to thirty seconds. The second sequential data 22 may be bio-signals related to an EEG, an EOG, and an EMG acquired through the nine channels from thirty-one seconds to sixty seconds which are time points subsequent to the first sequential data 21. Further, the sleep sensing data used for analysis may include, but is not limited to, ten pieces of sequential data in units of thirty seconds.

In this case, the dimension reduction sub-model 310 may extract one or more features corresponding to each channel using the first sequential data 21 as an input. Specifically, the dimension reduction sub-model may generate six features according to each of the six channels related to an EEG corresponding to zero seconds to thirty seconds, generate two features corresponding to each of the two channels related to an EOG corresponding to the time period, and generate one feature corresponding to the one channel related to an EMG corresponding to the time period. In other words, the dimension reduction sub-model 310 may generate nine features corresponding to each channel when the first sequential data 21 included in the sleep sensing data is input. Also, the dimension reduction sub-model 310 may generate an integrated feature by integrating one or more features, that is, nine features. The integrated feature is obtained by integrating various bio-signals acquired through the plurality of channels for the same time and may mean a feature that represents a sleep environment corresponding to a specific time point. In other words, one integrated feature may mean a feature (embedding) corresponding to one piece of sequential data. Also, the dimension reduction sub-model 310 may generate an integrated feature corresponding to the second sequential data 22 using the second sequential data 22 related to the sleep environment of a time point subsequent to the first sequential data 21 as an input.

For example, a model that extracts a feature corresponding to each channel in the dimension reduction sub-model 310 may be a residual 1-dimensional convolutional neural network (Res1DCNN), and a model that extracts an integrated feature by integrating channel-specific features may also be a Res1DCNN. In other words, the dimension reduction sub-model 310 may be a multi-residual 1-dimensional convolutional neural network (MultiRes1DCNN) but is not limited thereto.

In an embodiment, a plurality of dimension reduction sub-models of the present disclosure may be provided to perform integrated feature extraction operations corresponding to one or more pieces of sequential data in parallel. When the plurality of dimension reduction sub-models are provided to perform one or more integrated feature extraction operations in parallel using each piece of sequential data as an input, the processing rate can be increased.

As described above, when sleep sensing data including one or more pieces of sequential data divided by the predetermined unit time is used as an input, the dimension recovery sub-model 330 may generate one or more integrated features corresponding to each of the pieces of sequential data. For example, when ten sequences are analyzed, ten integrated features may be acquired.

Also, the dimension recovery sub-model 330 may output sleep analysis information using the integrated features as an input.

As a specific example, the dimension recovery sub-model 330 may include an RNN. The dimension recovery sub-model 330 may analyze integrated features of a plurality of sequences together using the plurality of integrated features acquired as analysis results of the plurality of sequences as an input and may output sleep analysis information on each of the plurality of sequences. The output sleep analysis information may include, but is not limited to, sleep stage information on each sequence and certainty information on the sleep stage information.

For example, when sleep analysis information is acquired using integrated features extracted from ten sequences, integrated features extracted through ten MultiRes1DCNN operations may be input to the RNN, and sleep stage information for each of the ten sequences and certainty information on the sleep stage information may be acquired from the output, but sleep analysis information is not limited thereto.

As another example, the dimension recovery sub-model 330 may include one or more RNNs and may be a model that outputs prediction information on a second timestamp through the one or more RNNs using prediction information on a first timestamp as an input. In this case, the first timestamp may be an earlier time point than the second timestamp. Specifically, prediction information predicted through a first RNN of the dimension recovery sub-model may be transferred to a second RNN of the next timestamp, and prediction information predicted through the second RNN may be transferred to a third RNN of the next timestamp. In this process, the one or more attention modules may determine a time point of a feature to be focused on through association information between time steps. In other words, the dimension recovery sub-model 330 determines a time point of a change factor to be focused on through the one or more attention modules 320 in a process of repeatedly predicting prediction information through the one or more RNNs and thus may output prediction information on associations between time points of various factors.

In other words, integrated features output through the dimension reduction sub-model 310 may be transferred to the one or more attention modules 320. In this case, the one or more attention modules 320 may predict an attention weight for sleep analysis information corresponding to each of the integrated features. The one or more attention modules 320 may emphasize an element to be focused on between input data and output data by giving an attention weight to each feature of time-series input data (i.e., sleep sensing data).

In other words, the sleep analysis model including the dimension reduction sub-model 310, the one or more attention modules 320, and the dimension recovery sub-model 330 of the present disclosure does not output sleep analysis information (i.e., prediction information on a sleep stage) by only considering a time point related to one piece of sequential data but may output sleep analysis information reflecting pieces of sequential data of previous time points.

For example, when a medical specialist or a sleep technologist actually reads a polysomnography result, not only one sequence corresponding to thirty seconds but also previous sequences may be taken into consideration for reading. In other words, when prediction information on a sleep stage is predicted based on a single sequence without considering associations with previous sequences, there is a risk of lack of accuracy.

Through the above-described process, the sleep analysis model of the present disclosure can predict a sleep stage by considering features of previous and subsequent sequential data in a process of predicting sleep analysis information corresponding to one piece of sequential data. Accordingly, it is possible to ensure accuracy and reliability of the predicted sleep analysis information.

According to an embodiment of the present disclosure, the sleep analysis model may perform transfer learning. For example, accuracy in predicting a sleep stage of patients suffering from sleep disorders may be lower than accuracy in predicting a sleep stage of normal people. For example, when a sleep stage is predicted using a neural network based on sleep sensing data acquired from patients suffering from sleep apnea, the accuracy may be lower than accuracy in predicting a sleep stage of users suffering from no sleep disorder by about 10% to 15%.

In general, it is difficult to clearly determine whether a user has a sleep disorder before performing a polysomnography. Accordingly, to generate a sleep analysis model that provides sleep analysis information corresponding to sleep sensing data of users suffering from a sleep disorder and users suffering from no sleep disorder, sleep sensing data related to users suffering from a sleep disorder may be additionally required. In other words, it is necessary to generate training data for training a sleep analysis model (i.e., a neural network model) from a plurality of pieces of sleep sensing data of a plurality of users suffering from a sleep disorder, which may result in a great deal of time and cost in training the neural network model with datasets.

Accordingly, the sleep analysis model of the present disclosure may perform transfer learning to output sleep analysis information corresponding to sleep sensing data which corresponds to another domain (e.g., users suffering from a sleep disorder). Specifically, the sleep analysis model may perform transfer learning to apply an algorithm trained in a source domain to a target domain. Here, the source domain may be related to sleep sensing data of users suffering from no sleep disorder, and the target domain may be related to sleep sensing data of users suffering from a sleep disorder. Transfer learning may mean, for example, recalibrating and using weights of a model trained in the source domain to suit the target domain. According to an additional embodiment, a sleep analysis model of the present disclosure may learn data through domain adaptation without a label related to a correct answer. In other words, the sleep analysis model may be improved in learning rate and performance through transfer learning and domain adaptation using a small amount of training data of various domains (e.g., users suffering from a sleep disorder or users suffering from no sleep disorder).

Accordingly, the sleep analysis model of the present disclosure can operate well not only for users suffering from no sleep disorder but also for users suffering from a sleep disorder. That is, the sleep analysis model may output solid sleep analysis information corresponding to sleep sensing data of a user suffering from a sleep disorder. In other words, the sleep analysis model may provide automated sleep analysis information that is robust to users having a sleep disorder.

According to an embodiment of the present disclosure, sleep analysis information output by the sleep analysis model may include prediction certainty information. The sleep analysis information may include prediction certainty information corresponding to each of one or more pieces of sequential data.

Specifically, the sleep analysis model may use sleep sensing data including one or more pieces of sequential data as an input to output sleep stage information corresponding to each piece of the sequential data. In this case, the sleep analysis model may predict a score (i.e., softmax) corresponding to each of the one or more sleep stages using one piece of the sequential data as an input and generate sleep stage information based on the score predicted according to each sleep stage. When prediction information on a sleep stage is predicted through the sleep analysis model, the processor 170 may generate prediction certainty information from score values contributing to calculation of the prediction information.

For example, when the first sequential data 21 acquired through ten channels from zero seconds to thirty seconds with regard to the user's sleep is used as an input, the sleep analysis model may predict a score corresponding to each of the one or more sleep stages according to the first sequential data 21. As a specific example, the sleep analysis model may predict a score of wake to be 2 points, a score of N1 to be 10 points, a score of N2 to be 80 points, a score of N3 to be 7 points, and a score of REM to be 1 point according to the first sequential data 21 and determine N2 as sleep stage information corresponding to the first sequential data 21 based on the largest score value. In other words, prediction information that the user's sleep stage corresponds to N2 sleep stage from zero seconds to thirty seconds may be generated. In this case, the processor 170 may determine prediction certainty information to be “80” based on the score contributing to prediction of N2 sleep stage. For example, large prediction certainty information may indicate that a sleep stage predicted through the artificial neural network has high accuracy (or reliability). The above-described details of sequential data, score values, sleep stages, and prediction certainty information are merely examples, and the present disclosure is not limited thereto.

In other words, the processor 170 may provide prediction information on sleep stages each corresponding to time points within the user's sleep and prediction certainty information corresponding to the prediction information on each sleep stage. In this case, the prediction certainty information may be used as an indicator for determining how reliable an output (i.e., prediction information on a sleep stage) of the artificial neural network (i.e., the sleep analysis model). In other words, prediction certainty information on prediction of sleep stage for each time point is provided together with sleep stage information such that sleep stage information can be reliably used.

According to an embodiment of the present disclosure, the processor 170 may modify prediction certainty information output by the sleep analysis model through temperature scaling. For example, using an artificial neural network in a medical field may show a risk not resulting from low average accuracy but resulting from operation uncertainty that the artificial neural network works appropriately in some situations but not in others. Accordingly, when prediction reliability information is generated only using a softmax value which is an output of a final output layer of the artificial neural network, it may be difficult to ensure reliability of prediction certainty information. For example, when the neural network is overconfident of its own prediction, there is a risk of delivering inaccurate information to users who refer to the prediction certainty information.

Accordingly, the processor 170 may perform an operation of brining the degree of confidence to which the model ensures a prediction and actual accuracy to the same level through a temperature scaling process. Specifically, when a logit vector Z is given using a single scalar parameter T, certainty prediction may be as follows.

$$\text{?} = \text{?} {( {z}_{i} / T )}^{( k )}$$

$$\text{?} \text{indicates text missing or illegible when filed}$$

When T increases, probability value \hat{q}ᵢ may approximate 1/K which represents a maximum uncertainty. In other words, according to the present disclosure, a prediction certainty may be modified through temperature scaling of performing optimization by minimizing the negative log likelihood (NLL) of a validation set. In this case, the temperature scaling may have influence not on the accuracy of the model but on the calibration.

In other words, it is possible to ensure the reliability of prediction certainty information that is used as a determination indicator of the user through certainty modification based on temperature scaling.

According to an embodiment of the present disclosure, the processor 170 may generate relationship information between prediction certainty information and sleep stage information. Also, the processor 170 may update sleep analysis information based on the relationship information.

Specifically, sleep analysis information including sleep stage information and prediction certainty information may be generated at every time point. As a specific example, sleep analysis information 400 may include sleep stage information and prediction certainty information generated corresponding to each time point as shown in FIG. 7. For example, first sleep analysis information 401 may be generated corresponding to the first sequential data 21 related to bio-signals acquired through the nine channels during a sleep from zero seconds to thirty seconds. The first sleep analysis information 401 may include information that the user's sleep corresponds to N1 at a time point corresponding to the first sequential data and prediction certainty information is 90%.

In this case, the processor 170 may generate relationship information by considering prediction certainty information and sleep stage information on the entire sleep period. For example, when the overall average of the prediction certainty information is predicted to be relatively low and the number of changes in sleep stage information during the entire sleep is larger than a predetermined change threshold, the processor 170 may generate relationship information that there are frequent changes in the sleep stage when prediction certainty is low. As another example, when the user is determined to have a disorder related to sleep apnea through a sleep stage ratio of the user's sleep and the overall average of the generated prediction certainty information is relatively low, the processor 170 may generate relationship information that sleep apnea is associated with low prediction certainty information. In other words, relationship information that a user having low prediction certainty is highly likely to have a sleep disorder may be generated. The above-described details of relationship information are merely examples, and the present disclosure is not limited thereto.

In other words, the processor 170 may generate relationship information between prediction certainty information and sleep stage information and update sleep analysis information based on the generated relationship information. This may allow provision of sleep analysis information based on new relationship information between a sleep pattern according to a change in the sleep stage and prediction certainty. Accordingly, the processor 170 may provide insight on the new relationship other than a generally-known pattern of sleep analysis to the user (e.g., a medical specialist).

According to an embodiment of the present disclosure, the processor 170 may generate feature map information based on the sleep analysis information. Specifically, the processor 170 may generate feature map information by visualizing attention weights for the sleep analysis information according to each integrated feature through the one or more attention modules.

As a specific example, from sleep sensing data of the user acquired according to a first time point (e.g., initial one minute of the user's sleep after going to bed), the computing device 100 may generate prediction information that a sleep stage of the user during the one minute corresponds to N3 sleep stage. Also, the computing device 100 may acquire an attention weight of each input element for sleep analysis information (i.e., the prediction information indicating that the sleep stage of the user at the time point corresponds to N3 sleep stage) corresponding to the first time point from the one or more attention modules. The computing device 100 may generate feature map information by visualizing the acquired element-specific attention weights.

In other words, the computing device 100 may visualize a basis of judgement for a process of generating prediction information on time-series input data (i.e., sleep sensing data) using the one or more attention modules. In other words, which signal at which time point (i.e., which sleep sensing data) plays an important role in determining a sleep stage of each time point may be visualized and provided through attention weight values. For example, the feature map information may be generated to provide visualized information in a meaningful form by differently displaying pixels based on signals or information that attracts attention through attention weights.

In general, it is difficult to understand an internal algorithm of an artificial neural network. In other words, it is difficult to know on what basis an output related to input data is generated. This acts as a risk resulting from uncertainty of the artificial neural network, and thus it may be difficult to use the artificial neural network in an actual medical field.

The processor 170 of the present disclosure visualizes a pattern of a signal that plays an important role in predicting a sleep stage at each time point and provides the visualized pattern, and thus it is possible to visualize on what basis a neural network model makes a prediction or determination. Accordingly, it is possible to precisely verify validity of prediction information output by the sleep analysis model of the present disclosure such that collaborative synergy with a user (e.g., a medical specialist) can be maximized.

A method performed by a processor to acquire object state information based on wireless communication will be described in detail below.

According to an embodiment, the processor 170 may determine to transmit a wireless signal through the transmission module 150. The wireless signal transmitted by the transmission module 150 may include an OFDM signal and may be divided and transmitted or received by a plurality of subcarriers corresponding to one or more antennas. For example, the wireless signal may be a WiFi-based OFDM signal, and the transmission module 150 may be a WiFi transmitter.

Also, the processor 170 may receive the wireless signal through the reception module 160. Specifically, the processor 170 may receive the transmitted wireless signal through the reception module 160 provided at a predetermined separation distance from the transmission module 150. The predetermined separation distance may mean an area in which an object is active or present. For example, the predetermined separation distance between the transmission module 150 and the reception module 160 may indicate an area in which the user takes a sleep. The wireless signal received through the reception module 160 is a wireless signal that has passed through a channel corresponding to the predetermined separation distance and may include information representing a characteristic of the channel.

According to an embodiment of the present disclosure, the processor 170 may acquire channel state information from the wireless signal. The channel state information may be information representing the characteristic of the channel related to an area in which the object for acquiring state information through the wireless signal is present and may be predicted based on the wireless signal transmitted by the transmission module and the wireless signal received by the reception module.

Specifically, the wireless signal transmitted by the transmission module 150 may pass through the specific channel (i.e., an area in which the user is present) and may be received by the reception module 160. In this case, the wireless signal may be transmitted through a plurality of subcarriers each corresponding to multiple paths. Accordingly, the wireless signal received through the reception module 160 may reflect movement of the object. The processor 170 may acquire the channel state information on the channel characteristic that the wireless signal has experienced while passing through the channel (i.e., an area in which the object is present) from the received wireless signal. The channel state information may have an amplitude and a phase. In other words, the processor 170 may acquire the channel state information on the characteristic of an area between the transmission module 150 and the reception module 160 (i.e., an area in which the object is present) based on the wireless signal transmitted by the transmission module 150 and the wireless signal received by the reception module 160 (i.e., a signal reflecting movement of the object).

According to an embodiment, the transmission module 150 and the reception module 160 may transmit or receive an OFDM signal through one or more antennas. For example, when each of the transmission module 150 and the reception module 160 includes three antennas, channel state information on a total of 192 (i.e., 3×64) channels may be acquired at each frame through the three antennas and 64 subcarriers. The detailed numbers of antennas and subcarriers are merely examples, and the present disclosure is not limited thereto.

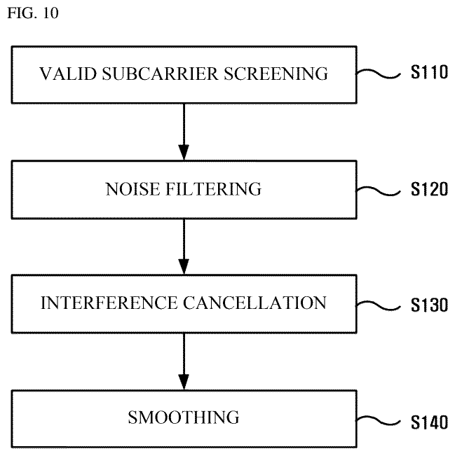

According to an embodiment of the present disclosure, the processor 170 may preprocess the channel state information. In the present disclosure, as shown in FIG. 10, preprocessing performed by the processor 170 may include preprocessing related to valid subcarrier screening (S110), preprocessing related to noise filtering (S120), preprocessing related to interference cancellation (S130), and preprocessing related to smoothing (S140).

Channel state information may be predicted corresponding to each of the plurality of subcarriers. Preprocessing channel state information may mean preprocessing a plurality of pieces of channel state information.

Specifically, the processor 170 may perform preprocessing to screen a valid subcarrier (S110). A wireless signal of the present disclosure may be data acquired in time series. Preprocessing for screening a valid subcarrier may be intended to screen a subcarrier including a large amount of information on movement of an object among a plurality of subcarriers extracted from one frame of a wireless signal.

As a specific example, An interval of 312.5 kHz may be provided between subcarriers of an OFDM signal. One frame of a 20 MHz OFDM signal includes sixty-four subcarriers, and there is a difference of 312.5 kHz in center frequency between the subcarriers. Accordingly, the subcarriers may experience different wireless channels. When a center frequency of each subcarrier s is fₛ, channel state information Hₛ(fₛ, t) obtained from subcarriers s may be represented as follows.

$${H}_{s} ( {f}_{s} , t ) = {\sum }_{i = 1}^{N} {A}_{i} \text{?}$$

$$\text{?} \text{indicates text missing or illegible when filed}$$

Here, N is the number of multi-paths that the subcarriers s experience, Aᵢ is signal attenuation in an i^{th} path, and Tᵢ(t) is a propagation delay occurring in the i^{th} path at a time t.

As seen in the Equation, the center frequency varies depending on the subcarriers, and thus a degree of phase difference of a signal caused by multi-paths may vary. Accordingly, information including information on movement of an object may vary.

For example, at a specific fₛ, a change in channel state information caused by chest movement (about 5 mm to 12 mm) of the user may be small. However, a subcarrier u having a different center frequency, experiences a different channel at a center frequency of fᵤ, and thus channel state information caused by chest movement may be large. Accordingly, a bio-signal can be accurately measured. In other words, channel state information on a specific subcarrier may bring a meaningful result for measuring a bio-signal (i.e., a more accurate measurement result) according to an area in which the user is present.

Therefore, the processor 170 may perform preprocessing to screen a subcarrier including a large amount of information on movement of the object (i.e., a valid subcarrier) among the plurality of subcarriers extracted from each frame of the wireless signal.

The processor 170 may perform frequency conversion on the plurality of pieces of channel state information. The frequency conversion means converting an input signal into frequency components and may include, for example, fast Fourier transform (FFT), discrete Hilbert transform (DHT), discrete wavelet transform (DWT), etc. The above-described details of frequency conversion are merely examples, and the present disclosure may further include various types of conversion for converting an input signal into frequency components. Also, the processor 170 may identify one or more first subcarriers having a conversion value within a predetermined threshold range based on results of the frequency conversion. The predetermined threshold range may be a general rate range of a target bio-signal. For example, a general breathing rate of people is ten to thirty times per minute, and the processor 170 may identify one or more first subcarriers corresponding to frequency bins within the range as FFT results. Also, the processor 170 may screen a valid subcarrier based on an energy level of each of the one or more first subcarriers. As a specific example, the processor 170 may compare the total sum of energy levels of all subcarriers with the energy level of each of the one or more first subcarriers within the rate range of a target bio-signal. The processor 170 may screen a subcarrier having the largest difference from the total sum of all the subcarrier energy levels as a valid subcarrier.

In other words, the processor 170 may identify subcarriers within the rate range of a target bio-signal through frequency conversion and screen a subcarrier having a high energy level as a valid subcarrier among the identified subcarriers (i.e., the one or more first subcarriers). Accordingly, a subcarrier including a large amount of information on movement of the object is screened as a valid subcarrier, and thus it is possible to increase accuracy in measurement (i.e., accuracy of output object state information) and also reduce noise.

The processor 170 may perform preprocessing related to noise filtering (S120). The processor may perform noise filtering on the wireless signal. For example, channel state information acquired from the screened valid subcarrier may include noise occurring at a time point at which the reception module 160 receives the wireless signal. A noise value of the wireless signal randomly varies and thus may correspond to a high frequency in the frequency domain. Therefore, to remove noise, the processor 170 may remove high-frequency components using a low-pass filter or a band-pass filter. In other words, the processor 170 may remove high-frequency components by applying at least one of a low-pass filter or a band-pass filter to the wireless signal received through the reception module 160.

In this case, a cutoff frequency of the low-pass filter or the band-pass filter may vary depending on the type of movement of an object to be measured. For example, in the case of measuring breathing of a person, frequency components deviating from the general breathing rate range of people, ten to twenty-five times per minute, may be handled as noise, and frequencies outside the range may be determined as cutoff frequencies. The above-described details related to the type of movement of an object, frequency components, and setting of cutoff frequencies are merely examples, and the present disclosure is not limited thereto.

The processor 170 may perform preprocessing related to interference cancellation (S130). The processor 170 may perform interference cancellation on the plurality of pieces of channel state information. Specifically, the processor 170 may reduce influence of interference by applying a Hampel filter to the plurality of pieces of channel state information.

For example, a value of channel state information may be notably changed by a wireless signal transmitted by another type of wireless device that shares a channel. Due to such interference, the value of the channel state information may have a large difference before and after the interference. The Hampel filter may be a filter that converts a signal value of a specific time into a median value of previous and subsequent signal values when the signal value of the specific time has a certain difference value or more with values of previous and subsequent times.

As a specific example, when a Hampel filter having a moving window size of 2K+1 and the number of standard deviations of n_sigma is applied to a vector x, a median absolute deviation (MAD), MAD=1.4826median(|[x_{n-K}, x_{n-K+1}, . . . , x_{n+K}]-median([x_{n-K}, x_{n-K+1}, . . . , x_{n+K}])|), is predicted with respect to xₙ, an n^{th} sample of the vector x. Then, when xₙ deviates from median([x_{n-K}, x_{n-K+1}, . . . , x_{n+K}])+n_signma*MAD range, xₙ is replaced with median([x_{n-K}, x_{n-K+1}, . . . , x_{n+K}]), and this is repeated for all values of n.

Therefore, when the Hampel filter having a moving window size of 2K+1 and the number of standard deviations of n_sigma is applied to channel state information Hₛ(fₛ, tₙ) which is the channel state information Hₛ(fₛ, t)=Σₜ₋ᵢ^{N}, Aᵢe^{-j2πf}^{ₛ}^{T}^{ᵢ}^{(t)} of the subcarrier s at the time tₙ, the MAD is defined as MAD=1.4826median(|[Hₛ(fₛ, t_{n-K}), Hₛ(fₛ, t_{n-K+1}), . . . , Hₛ(fₛ, t_{n+K})]-median([Hₛ(fₛ, t_{n-K}), Hₛ(fₛ, t_{n-K+1}), . . . , Hₛ(fₛ, t_{n+K})])|), and thus an appropriate range may be predicted as median([Hₛ(fₛ, t_{n-K}), Hₛ(fₛ, t_{n-K+1}), . . . , Hₛ(fₛ, t_{n+K})]±3MAD. In other words, when the channel state information Hₛ(fₛ, tₙ) of the subcarrier s at the time tₙ deviates from the range, the channel state information Hₛ(fₛ, tₙ) may be considered to be an interference and replaced with median([Hₛ(fₛ, t_{n-K}), Hₛ(fₛ, t_{n-K+1}), . . . , Hₛ(fₛ, t_{n+K})].

In other words, the processor 150 may apply the Hampel filter to the plurality of pieces of channel state information and thereby convert a signal value into a median value when the signal value has a large difference before and after interference. Accordingly, it is possible to reduce influence of interference by minimizing the amount of difference.

The processor 150 may perform preprocessing related to smoothing (240). The processor 150 may perform smoothing on the channel state information that has undergone noise cancellation. Specifically, the processor 150 may modify channel state information values by applying a Savitzky-Golay filter to the channel state information that has undergone noise cancellation. Smoothing may mean smoothly modifying a signal to clearly extract periodicity, and the Savitzky-Golay filter may mean a filter that causes a polynomial function to regress to a channel state information value to modify a value.

As a specific example, when a Savitzky-Golay filter having a moving window size of 2K+1 and a polynomial coefficient of m is applied to a vector x, a regression analysis may be performed on an m^{th}-order polynomial aₘx^{m}+aₘ₋₁x^{m-1}+ . . . +a₀ using 2K+1 samples including xₙ which is an n^{th} sample of the vector x to predict aₘ, aₘ₋₁, . . . , and a₀, and then aₘ, aₘ₋₁, . . . , and a₀ may be inserted into aₘxₙ^{m}+aₘ₋₁xₙ^{m-1}+ . . . +a₀ obtained by inserting xₙ into the polynomial. Subsequently, the same operation may be repeated for all values of n.

In other words, the processor may modify channel state information values by applying the Savitzky-Golay filter to the channel state information, thereby reducing influence of interference.

According to an embodiment of the present disclosure, the processor 170 may acquire object state information from the preprocessed channel state information. The object state information may include information on movement and bio-signals (e.g., a heart rate or respiration) of the object. Specifically, the processor 170 may identify a rotation period on a complex plane based on the channel state information. The preprocessed channel state information acquired through the above-described preprocessing process may be displayed in an area of the complex plane. A propagation delay varies depending on movement of the object in an environment in which the transmission module 150 and the reception module 160 are disposed, and such a variation of the propagation delay may be reflected in the channel state information. In other words, the propagation delay varies depending on movement of the object, and such a variation of the propagation delay may be represented by a phase difference of the channel state information. Such a phase difference may cause a large movement (or change) on the complex plane even with a slight movement of the object. For example, a change in the phase difference may vary depending on a wavelength of the wireless signal. 2.4 GHz and 5 GHz signals generally used in WiFi may have wavelengths of 12.5 cm and 6 cm, respectively. Accordingly, even with a change of several centimeters to several millimeters, the phase difference may change relatively large and make a large change in the complex plane. In other words, according to the present disclosure, it is possible to generate object state information on subtle movement of the object.

The channel state information on the present disclosure is acquired from the wireless signal that is acquired in time series. When the channel state information is repeatedly acquired, a displayed value of the channel state information may vibrate counterclockwise on the complex plane according to periodic movement of the object (e.g., respiration). For example, the displayed value of the channel state information may draw a circle or an arc on the complex plane while vibrating depending on the size of the movement of the object. The processor 170 may identify a rotation period on the complex plane through the repeatedly acquired channel state information and acquire object state information based on the identified rotation period. Specifically, the processor 170 may identify a rotation period based on at least one of a size or ratio of the preprocessed channel state information and acquire object state information based on the identified rotation period.

According to an embodiment of the present disclosure, the processor 170 may identify a rotation period based on the size of the channel state information. The size of the channel state information may periodically change due to a phase difference occurring on the complex plane. Accordingly, the processor 170 may perform frequency conversion on the preprocessed channel state information to identify a signal periodically vibrating based on the time axis. The frequency conversion means converting an input signal into frequency components and may include, for example, FFT, DHT, DWT, etc. The above-described details of frequency conversion are merely examples, and the present disclosure may further include various types of conversion for converting an input signal into frequency components. Also, the processor 170 may identify a rotation period based on results of the frequency conversion.

The processor 170 may identify channel state information having a highest energy level as the results of the frequency conversion and determine a period corresponding to the identified channel state information as a period related to movement of the object (e.g., breathing of the user). According to an embodiment, the processor 170 may predict an autocorrelation coefficient by sliding the signal on the time axis and determine a first period in which the autocorrelation coefficient becomes the largest as a period related to movement of the object within a rate range of target movement of the object (e.g., in the case of a breathing rate, ten to thirty times per minute).

According to another embodiment of the present disclosure, the processor 170 may identify a rotation period based on the ratio of the channel state information. Identifying a rotation period based on the ratio of the channel state information may be a method used, for example, when the transmission module or the reception module includes a plurality of antennas. In other words, when channel state information is received from a plurality of antennas, identifying a rotation period based on a ratio of the channel state information may mean identifying a rotation period using the ratio of the channel state information. When a rotation period is identified based on the ratio of the channel state information and object state information on the object is acquired using the identified rotation period, it is possible to acquire object state information that is more accurate than the channel state information.

Specifically, channel state information extracted from a frame of an OFDM signal related to a wireless signal of the present disclosure may include noise. For example, since the transmission module 150 and the reception module 160 employ different oscillators, an error, such as a center frequency offset (CFO) and a sampling frequency offset (SFO), may occur due to a difference in precision between the oscillators. Accordingly, unintended noise may be included in channel state information. Since the error of the CFO or SFO remarkably varies depending on circumstances, such noise may be difficult to remove. In other words, it may be difficult to predict and correct noise because an oscillator varies depending on used equipment and an actual operation speed of an oscillator varies due to a temperature and the like.

Accordingly, the processor 170 may correct the foregoing noise using channel state information received through two or more antennas. Specifically, when channel state information is received by one reception module 160 from two or more antennas, the same oscillator is shared, and thus the same amount of noise may occur due to an oscillator difference with the transmitter such as a CFO or an SFO. In other words, when the both-hand sides are divided by the same amount of error, the noise is reduced to its lowest term such that a precise ratio value of channel state information can be acquired.

The ratio of channel state information may be considered a value obtained by performing translation, constant multiplication, and complex inversion on channel state information H on the complex plane and may maintain an original feature even after such calculation.

Accordingly, like identifying a rotation period based on the size of channel state information, the processor 170 may identify a period of moving on the complex plane using frequency conversion or the autocorrelation coefficient and determine the identified period as a period related to movement of the object.

In other words, the processor 170 may identify a rotation period related to movement of the object on the complex plane through channel state information acquired in time series and acquire object state information through the rotation period. For example, the processor 170 may identify a rotation period related to the user's breathing through channel state information and acquire a bio-signal related to the user's breathing through the rotation period.

In other words, the processor 170 may acquire object state information on monitoring information on the object based on wireless communication. In this case, the object state information may be related to movement or a bio-signal of the user. In other words, the processor 170 may acquire state information on movement and a bio-signal of the user using a method of not coming into contact with the user's body.

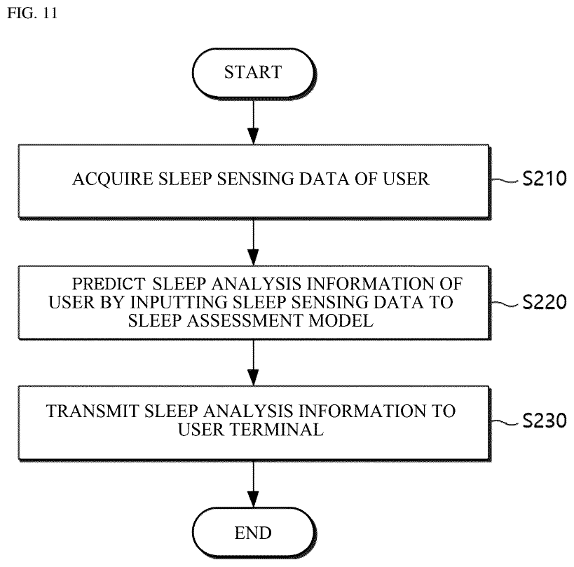

FIG. 11 is a flowchart illustrating a method of providing sleep analysis information based on sleep sensing data related to a sleep environment of a user according to one embodiment of the present disclosure.

According to an embodiment of the present disclosure, the method may include an operation S210 of acquiring sleep sensing data of a user.

According to an embodiment of the present disclosure, the method may include an operation S220 of inputting the sleep sensing data to a sleep assessment model to predict sleep analysis information on the user.

According to the embodiment of the present disclosure, the method may include an operation S230 of transmitting the sleep analysis information to a user terminal.

The above-described operations illustrated in FIG. 11 may be changed in order as necessary, and at least one operation may be skipped or added. In other words, the above-described operations are merely an embodiment of the present disclosure, and the scope of the present disclosure is not limited thereto.

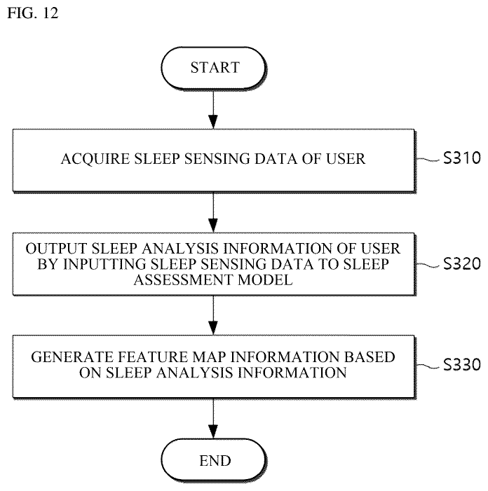

FIG. 12 is a flowchart illustrating a method of providing sleep-related analysis information according to one embodiment of the present disclosure.

According to an embodiment of the present disclosure, the method may include an operation S310 of acquiring sleep sensing data of a user.

According to an embodiment of the present disclosure, the method may include an operation S320 of outputting sleep analysis information on the user by processing the sleep sensing data as an input to a sleep assessment model.

According to an embodiment of the present disclosure, the method may include an operation S330 of generating feature map information based on the sleep analysis information.

The above-described operations illustrated in FIG. 12 may be changed in order as necessary, and at least one operation may be skipped or added. In other words, the above-described operations are merely an embodiment of the present disclosure, and the scope of the present disclosure is not limited thereto.

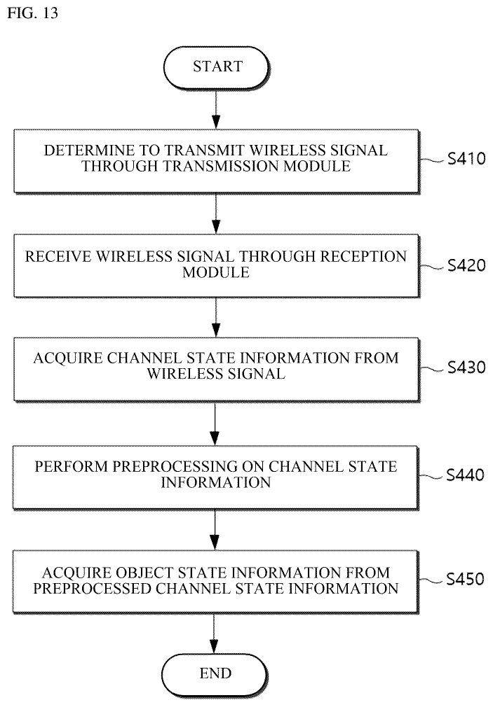

FIG. 13 is a flowchart illustrating a method of acquiring object state information based on wireless communication according to one embodiment of the present disclosure.

According to an embodiment of the present disclosure, the method may include an operation S410 of determining to transmit a wireless signal through a transmission module.

According to an embodiment of the present disclosure, the method may include an operation S420 of receiving a wireless signal through a reception module.

According to an embodiment of the present disclosure, the method may include an operation S430 of acquiring channel state information from the wireless signal.

According to an embodiment of the present disclosure, the method may include an operation S440 of preprocessing the channel state information.

According to an embodiment of the present disclosure, the method may include an operation S450 of acquiring object state information from the preprocessed channel state information.

The above-described operations illustrated in FIG. 13 may be changed in order as necessary, and at least one operation may be skipped or added. In other words, the above-described operations are merely an embodiment of the present disclosure, and the scope of the present disclosure is not limited thereto.

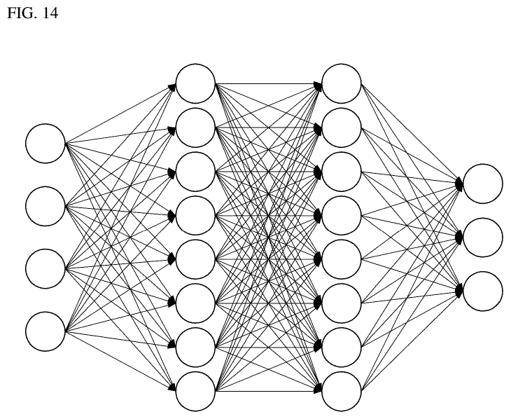

FIG. 14 is a schematic diagram illustrating one or more network functions according to one embodiment of the present disclosure.

Throughout the specification, a computation model, a network function, and a neural network may be used for the same meaning. The neural network may generally be configured with a set of interconnected calculation units which may be referred to as “nodes.” The “nodes” may also be referred to as “neurons.” The neural network includes one or more nodes. The nodes (or neurons) constituting the neural network may be connected to each other by one or more “links.”

In the neural network, one or more nodes connected through a link may relatively have a relationship of an input node and an output node. Concepts of the input node and the output node are relative so that any node which serves as an output node for one node may also serve as an input node for another node and vice versa. As described above, the relationship of an input node and an output node may be created around a link. One or more output nodes may be connected to one input node through a link and vice versa.

In the relationship of an input node and an output node connected through one link, a value of the output node may be determined based on data input to the input node. A node interconnecting the input node and the output node to each other may have a weight. The weight may be variable and may vary by a user or an algorithm to allow the neural network to perform a desired function. For example, when one or more input nodes are connected to one output node through respective links, the output node may determine an output node value based on values input to the input nodes connected to the output node and weights set for the links each corresponding to the input nodes.

As described above, in the neural network, one or more nodes are connected to each other through one or more links to have a relationship of an input node and an output node. A characteristic of the neural network may be determined according to the number of nodes and links, correlations between the nodes and the links, and weights each assigned to the links in the neural network. For example, when there are two neural networks in which the same number of nodes and links are present and weights of the links are different, the two neural networks may be recognized to be different.

The neural network may include one or more nodes. Some of the nodes constituting the neural network may constitute one layer based on the distance from an initial input node. For example, a set of nodes having a distance of n from the initial input node may constitute an n^{th}-order layer. The distance from the initial input node may be defined by the minimum number of links that it is necessary to go though from the initial input node to reach to a corresponding node. However, the definition of a layer is arbitrarily provided for description and the order of a layer in the neural network may be defined in a different way than that described above. For example, a layer of nodes may be defined by the distance from a final output node.

Initial input nodes may be one or more nodes to which data is directly input without passing through a link in the relationship with other nodes among the nodes in the neural network. Alternatively, in the neural network, initial input nodes may be nodes that do not have another input node connected by a link in the relationship between nodes around a link. Similarly, final output nodes may be one or more nodes that do not have another output node in the relationship with other nodes among the nodes in the neural network. Further, hidden nodes may be nodes that constitute the neural network other than the initial input node and the final output node. In the neural network according to an embodiment of the present disclosure, the number of nodes of the input layer may be equal to the number of nodes of the output layer, and the number of nodes may be reduced and then increased from the input layer to the hidden layer. Also, in a neural network according to another embodiment of the present disclosure, the number of nodes of the input layer may be smaller than the number of nodes of the output layer, and the number of nodes may be reduced from the input layer to the hidden layer. Further, in a neural network according to still another embodiment of the present disclosure, the number of nodes of the input layer may be larger than the number of nodes of the output layer, and the number of nodes may increase from the input layer to the hidden layer. A neural network according to yet another embodiment of the present disclosure may be a neural network that is a combination of the above-described neural networks.

A deep neural network (DNN) may be a neural network including a plurality of hidden layers in addition to the input layer and the output layer. When the DNN is used, latent structures of data may be identified. In other words, it is possible to identify latent structures of photos, text, video, voice, and music (e.g., which objects are in a photo, what is the content and emotion of text, what is the content and emotion of voice, etc.). The DNN may include a CNN, an RNN, an autoencoder, a generative adversarial network (GAN), a restricted Boltzmann machine (RBM), a deep belief network (DBN), a Q network, a U network, a Siamese network, etc. The foregoing DNNs are merely examples, and the present disclosure is not limited thereto.

The neural network may be trained by at least one of supervised learning, unsupervised learning, and semi-supervised learning methods. Training of the neural network is to minimize an error of the output. Training of the neural network is a process of updating a weight of each node in the neural network by repeatedly inputting training data to the neural network, predicting an output of the neural network for the training data and an error of the target, and backpropagating the error of the neural network from the output layer of the neural network to the input layer. In the case of supervised learning, training data obtained by labeling each piece of training data with a correct answer (i.e., labeled training data) is used, and in the case of unsupervised learning, each piece of training data may not be labeled with a correct answer. In other words, for example, training data of supervised learning for data classification may be data obtained by labeling each piece of training data with a category. The labeled training data is input to the neural network, and an error may be predicted by comparing an output (category) of the neural network with the label of the training data. As another example, in the case of unsupervised learning for data classification, an error may be predicted by comparing training data which is an input with an output of the neural network. The predicted error is backpropagated in a reverse direction (i.e., a direction from the output layer to the input layer) in the neural network, and a connection weight of each node in each layer of the neural network may be updated by the backpropagation. A variation of the connection weight of each node which is updated may vary depending on a learning rate. The calculation of the neural network for the input data and the backpropagation of the error may constitute a learning epoch. Different learning rates may be applied depending on the number of learning epochs repeated by the neural network. For example, at the early stage of learning of the neural network, the neural network may quickly ensure a certain level of performance using a high learning rate to increase efficiency, and at the late stage of learning, a low learning rate may be used to increase accuracy.

In the learning of the neural network, the training data may generally be a subset of actual data (i.e., data to be processed using the trained neural network). Therefore, there may be a learning epoch that results in a reduced error of the training data but an increased error of the actual data. Overfitting is a phenomenon in which training data is excessively learned so that an error for real data is increased. For example, a phenomenon that a neural network which learns cats through yellow cats does not recognize a cat other than yellow cats as a cat may be a sort of overfitting. The overfitting may cause an increase in an error of the machine learning algorithm. Various optimization methods may be used to prevent such overfitting. To prevent overfitting, a method of increasing training data, regularization, a dropout method of omitting some nodes of a network during a training process, etc. may be used.

Throughout the specification, a computation model, a neural network, a network function, and a neural network may be used for the same meaning (hereinafter, collectively referred to as a “neural network”). A data structure may include a neural network. The data structure including the neural network may be stored on a computer-readable medium. The data structure including the neural network may also include data input to the neural network, weights of the neural network, a hyperparameter of the neural network, data acquired from the neural network, activation functions each related to nodes or layers of the neural network, and a loss function for learning of the neural network. The data structure including the neural network may include any of the configuration elements among the disclosed elements. In other words, the data structure including the neural network may include the entirety or any combination of data input to the neural network, weights of the neural network, a hyperparameter of the neural network, data acquired from the neural network, activation functions each related to nodes or layers of the neural network, and a loss function for learning of the neural network. Besides the foregoing elements, the data structure including the neural network may include any other information for determining a characteristic of the neural network. Also, the data structure may include all types of data used or generated in a computation process of the neural network and is not limited to the above description. The computer-readable medium may include a computer-readable recording medium and/or a computer-readable transmission medium. The neural network may be formed of a set of interconnected calculation units that are generally referred to as “nodes.” The nodes may also be called “neurons.” The neural network includes at least one or more nodes.

The data structure may include data input to the neural network. The data structure including the data input to the neural network may be stored in the computer-readable recording medium. The data input to the neural network may include training data input in the process of training the neural network and/or input data input to the neural network having completed learning. The data input to the neural network may include data having undergone preprocessing and/or data to be preprocessed. The preprocessing may include a data processing process for inputting data to the neural network. Accordingly, the data structure may include data to be preprocessed and data generated by the preprocessing. The above-described data structure is merely an example, and the present disclosure is not limited thereto.

The data structure may include a weight of the neural network (in the specification, a “weight” and a “parameter” may be used for the same meaning). Further, the data structure including the weight of the neural network may be stored on the computer-readable medium. The neural network may include a plurality of weights. The weight may be variable and varied by a user or an algorithm in order for the neural network to perform a desired function. For example, when one or more input nodes are connected to one output node by their own links, a value of the output node may be determined based on values input to the input nodes connected to the output node and parameters set for the links each corresponding to the input nodes. The above-described data structure is merely an example, and the present disclosure is not limited thereto.

As a non-limited example, weights may include weights varied in the neural network training process and/or weights of the neural network having completed learning. The weights varied in the neural network training process may include a weight at a time at which a training cycle starts and/or a weight varied during a training cycle. The weights of the neural network having completed learning may include weights of the neural network having completed the training cycle. Accordingly, the data structure including the weights of the neural network may include the data structure including the weights varied in the neural network training process and/or the weights of the neural network having completed learning. Therefore, it is assumed that the foregoing weights and/or a combination of weights are included in the data structure including the weights of the neural network. The above-described data structure is merely an example, and the present disclosure is not limited thereto.

The data structure including the weight of the neural network may be subjected to a serialization process and then stored on the computer-readable storage medium (e.g., a memory and a hard disk). The serialization may be a process of storing the data structure in the same or different computing devices and converting the data structure into a form that may be reconstructed and used later. The computing device may serialize the data structure and then transmit or receive the data through a network. The serialized data structure including the weights of the neural network may be reconstructed in the same or different computing devices through deserialization. The data structure including the weights of the neural network is not limited to the serialization. Further, the data structure including the weights of the neural network may include a data structure (e.g., in a non-linear data structure, a B-tree, a Trie, an m-way search tree, an Adelson-Velsky and Landis (AVL) tree, and a red-black tree) for improving efficiency of computation while minimally using resources of the computing device. The above description is merely an example, and the present disclosure is not limited thereto.

The data structure may include a hyperparameter of the neural network. The data structure including the hyperparameter of the neural network may be stored in the computer-readable medium. The hyperparameter may be a variable changed by a user. The hyperparameter may include, for example, a learning rate, a cost function, the number of times of repetition of the training cycle, weight initialization (e.g., setting of a range of a weight to be initialized), and the number of hidden units (e.g., the number of hidden layers and the number of nodes in the hidden layer). The above-described data structure is merely an example, and the present disclosure is not limited thereto.

The operations of the methods or algorithms described above in connection with embodiments of the present disclosure may be implemented directly by hardware, a software module executed by hardware, or a combination thereof. The software module may reside in a RAM, a ROM, an erasable programmable read-only memory (EPROM), an EEPROM, a flash memory, a hard disk, a detachable disk, a CD-ROM, or any form of computer-readable recording medium well known in the technical field to which the present disclosure pertains.

Components of the present disclosure may be embodied in the form of a program (or an application) and stored in a medium to be executed in combination with a computer which is hardware. The components of the present disclosure may be implemented by software programming or software elements, and similarly, embodiments may be implemented in a programming or scripting language, such as C, C++, Java, an assembler, etc., including data structures, processes, routine, or various algorithms which are combinations of other programming components. Functional aspects may be embodied as an algorithm executable by one or more processors.

Those of ordinary skill in the art will appreciate that various illustrative logical blocks, modules, processors, means, circuits, and algorithm steps described in connection with the embodiments disclosed herein may be implemented as electronic hardware, various types of programs or design codes (hereinafter, “software” for easy description), or a combination of all of them. To clearly describe the compatibility of the hardware and the software, various exemplary components, blocks, modules, circuit, and steps have been generally described above in connection with functions thereof. Whether the functions are implemented as hardware or software depends on design restrictions given to a specific application and an entire system. Those of ordinary skill in the art of the present disclosure may implement functions described in various ways with respect to each specific application, but it should not be analyzed that the implementation determination departs from the scope of the present disclosure.

Various exemplary embodiments presented herein may be implemented as manufactured articles using a method, an apparatus, or a standard programming and/or engineering technique. The term “manufactured article” includes a computer program, a carrier, or a medium which is accessible by any computer-readable device. For example, the computer-readable medium includes a magnetic storage device (e.g., a hard disk, a floppy disk, a magnetic strip, etc.), an optical disk (e.g., a CD, a DVD, etc.), a smart card, and a flash memory device (e.g., an EEPROM, a card, a stick, a key drive, etc.) but is not limited thereto. Also, various storage media presented herein include one or more device and/or other machine-readable media for storing information. The term “machine-readable media” include a wireless channel and various other media that can store, include, and/or carry instructions and/or data but is not limited thereto.

It will be appreciated that a specific order or a hierarchical structure of steps in the presented processes is one example of exemplary approaches. It will be appreciated that the specific order or the hierarchical structure of the steps in the processes within the scope of the present disclosure may be rearranged based on design priorities. Appended method claims provide elements of various steps in a sample order, but it does not mean that the method claims are limited to the presented specific order or hierarchical structure.

The description of the presented embodiments is provided so that those of ordinary skill in the art of the present disclosure use or implement the present disclosure. Various modifications to the embodiments will be apparent to those of ordinary skill in the art of the present disclosure. Generic principles defined herein may be applied to other embodiments without departing from the scope of the present disclosure. Therefore, the present disclosure is not limited to the embodiments presented herein but should be analyzed within the widest range which is consistent with the principles and new characteristics presented herein.

### Modes of the Invention

Related descriptions have been provided above in the Best Mode for Carrying Out the Invention.

### Industrial Applicability

The present disclosure can be used in a field in which a sleep state is predicted based on bio-signals of a user to provide diagnosis information.

1. A computing device for predicting a sleep state based on data measured in a sleep environment of a user, the computing device comprising:
a processor configured to receive sleep sensing data of the user and predict sleep analysis information on the user by inputting the sleep sensing data to a sleep assessment model;
a memory configured to store program codes executable by the processor; and
a network unit configured to transmit or receive data to or from a user terminal,
wherein the sleep sensing data includes information on breathing of the user obtained during a predetermined time period with regard to the sleep environment of the user, and
the sleep analysis information includes at least one of apnea severity information on a degree of apnea occurrence of the user and disorder prediction information on a probability of disorder occurrence of the user.

2. The computing device of claim 1, further comprising a sensor unit configured to acquire the sleep sensing data of the user,
wherein the sensor unit comprises:
at least one transmission module configured to transmit a radio wave of a specific frequency; and
a reception module configured to receive a reflected wave generated in response to the radio wave of the specific frequency, and
wherein the sensor unit acquires the sleep sensing data from the user in a contactless manner by detecting a phase difference or a frequency change according to a travel distance of the reflected wave.

3. The computing device of claim 1, wherein the processor receives a sleep diagnosis dataset including a plurality of pieces of sleep diagnosis data each corresponding to a plurality of users,
generates training input datasets by extracting information on the users' respiration, heart rates, and movement during the predetermined time period from each of the plurality of pieces of sleep diagnosis data,
generates training output datasets by extracting at least one of information on apnea indices, information on sleep states, and information on whether a disorder has occurred from each of the plurality of pieces of sleep diagnosis data,
generates labeled training datasets by matching the training input datasets with the training output datasets, and
generates the sleep assessment model by training one or more network functions using the labeled training datasets.

4. The computing device of claim 3, wherein, to generate the sleep assessment model, the processor inputs each of the training input datasets to the one or more network functions, derives errors by comparing output data predicted through the one or more network functions with the training output datasets corresponding to labels of the training input datasets, adjusts weights of the one or more network functions based on the errors using a backpropagation method, determines whether to stop training using verification data when the one or more network functions are trained for predetermined epochs or more, and tests performance of the one or more network functions using test datasets to determine whether to activate the one or more network functions.

5. The computing device of claim 1, wherein the sleep assessment model is a model for predicting the user's sleep state and probability of disorder occurrence based on the sleep sensing data and includes one or more network functions, which include a dilated convolutional neural network (CNN) for strengthening a long-term relationship without loss of input data.

6. The computing device of claim 5, wherein the dilated CNN strengthens the long-term relationship by expanding a receptive field of a filter related to inputs of the one or more network functions and maintains lengths of the inputs and outputs of the one or more network functions by adding zero-padding to the filter related to the inputs.

7. The computing device of claim 1, wherein the disorder prediction information includes prediction information on at least one of sleep disorders, mental disorders, brain disorders, and cardiovascular disorders, and
the processor derives a correlation between the sleep sensing data and the disorder prediction information based on a variation of the disorder prediction information that is output by changing the information on breathing for the predetermined time period included in the sleep sensing data of the user input to the sleep assessment model.

8. The computing device of claim 1, wherein the sleep sensing data includes information on movement and information on heart rate for the predetermined time period, and
the sleep analysis information includes sleep stage information on changes in one or more sleep states over time with respect to the sleep environment of the user.

9. The computing device of claim 8, further comprising a sensor unit including one or more environment sensing modules for acquiring indoor environment information including information on at least one of a body temperature of the user, an indoor temperature, an indoor humidity, an indoor sound, and an indoor brightness,
wherein the processor generates sleep degradation factor information based on the sleep stage information and the indoor environment information.

10. The computing device of claim 9, wherein the processor identifies a first time point at which quality of sleep of the user is degraded based on the sleep stage information,
identifies a singularity related to a variation of the indoor environment information corresponding to the first time point, and
generates the sleep degradation factor information based on the identified singularity.

11. The computing device of claim 10, wherein the processor identifies the singularity related to volatility of the indoor environment information based on whether a variation of each of the at least one piece of information included in the indoor environment information exceeds a predetermined threshold variation corresponding to the first time point.

12. The computing device of claim 9, further comprising an indoor environment setup unit configured to adjust at least one of temperature, the humidity, the sound, and the brightness to setup an indoor environment related to the sleep environment of the user,
wherein the processor generates an environment control signal for controlling the indoor environment setup unit based on the sleep degradation factor information.

13. The computing device of claim 1, wherein the processor generates healthcare information for improving the sleep environment and health of the user based on the sleep assessment information and determines to transmit the healthcare information to the user terminal of the user, and
the healthcare information includes at least one of eating habit information, exercise amount information, and optimal sleep environment information.

14. A method of predicting a sleep state and disorder based on data measured in a sleep environment of a user, which is performed by at least one processor of a computing device, the method comprising:
receiving, by the processor, sleep sensing data of the user;
inputting, by the processor, the sleep sensing data to a sleep assessment model to predict sleep analysis information on the user; and
determining, by the processor, to transmit the sleep analysis information to a user terminal of the user.

15. A non-transitory computer-readable medium having stored therein a computer program for causing a computing apparatus to execute the following operations of:
receiving sleep sensing data of the user;
inputting, by the processor, the sleep sensing data to a sleep assessment model to predict sleep analysis information on the user; and
determining, by the processor, to transmit the sleep analysis information to a user terminal of the user.
